(19) United States
(12) Patent Application Publication
(10) Pub. No.: US 2008/0219890 A1
(43) Pub. Date: Sep. 11, 2008

(54) SAMPLE LOADING AND RECOVERY

(75) Inventors: John Lawson, Petersham, MA (US); Parris Wellman, Reading, MA (US); Sepehr Kiani, Watertown, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: Helicos Biosciences Corporation, Cambridge, MA (US)

(21) Appl. No.: 12/043,116

(22) Filed: Mar. 5, 2008

Related U.S. Application Data
(63) Continuation-in-part of application No. 11/997,382, filed on Jan. 30, 2008.
(60) Provisional application No. 60/813,428, filed on Jun. 13, 2006.
(60) Provisional application No. 60/705,847, filed on Aug. 4, 2005.

(51) Int. Cl. B01L 3/00; G01N 1/10; C12M 1/00

(52) U.S. Cl. 422/81; 435/287.3; 435/288.4

(57) ABSTRACT

A apparatus and method for loading a sample into a microfluidic flow cell allows for more precise loading, reduced cross-contamination, and more efficient use of samples to be analyzed. The system for loading a sample into a flow cell includes a flow cell defining a plurality of individually isolated channels through which fluid can flow. The flow cell also defines an inlet port and an outlet port for each of the channels. The system also includes a base that defines a chamber for receiving the flow cell, a cover pivotally attached to the base, and a passive vacuum source for pulling a volume through the flow cell. The method of loading a sample includes inserting the flow cell into the sample loading apparatus, placing a sample in at least one of the wells of the loading block, activating a vacuum source fluidly coupled to the outlet ports to pull the sample into the channel, and optionally aspirating the unused sample from the well.

### CROSS-REFERENCE TO RELATED CASES

This application is a continuation-in part of U.S. application Ser. No. 11/997,382 filed Jan. 30, 2008 which is a National Stage Entry of international application PCT/US06/030824 filed on Aug. 4, 2006 which claims priority to and the benefit of Provisional U.S. Patent Application Ser. No. 60/705,847, filed Aug. 4, 2005, and also Provisional U.S. Patent Application Ser. No. 60/813,428, filed Jun. 13, 2006. The entirety of each of these patent applications is incorporated herein by reference.

### TECHNICAL FIELD

The invention relates generally to loading samples into microfluidic flow cells and also to recovery unused sample.

### BACKGROUND INFORMATION

Fluidic systems are used in a variety of technical areas including biochemical analysis, medical diagnostics, analytical chemistry, chemical synthesis, and environmental monitoring. Microfluidic systems provide certain advantages in acquiring chemical and biological information. For example, microfluidic systems permit complicated processes to be carried out using small amounts of reagents.

### SUMMARY OF THE INVENTION

The invention generally provides flow cells, particularly multi-channel flow cells, and methods and devices for loading and using flow cells in order to streamline the process of reaction and interrogation of biochemical assays at the microfluidic level. Flow cells of the invention are useful for conducting high-throughput, multiplexed chemical reactions.

Specifically, the invention also provides apparatus and methods for loading a sample into a microfluidic flow cell. The invention allows for more precise loading, reduced cross-contamination, and more efficient use of samples to be analyzed. Systems for loading a sample into a flow cell comprise an apparatus comprising a housing adapted to receive a flow cell, a cover adapted to secure the flow cell in the apparatus. In addition the loader can comprise at least one loading block for introducing a sample into microchannels in the flow cell, and optionally a vacuum source for pulling sample from the loading block into the microchannels. In addition, systems also may include a base that defines a chamber for receiving the flow cell, and a cover pivotally attached to the base.

Additionally, as shown in the attached Figures, the system can comprise a housing for containing the aforementioned elements and a cover that articulates with the housing and whereby the cover secures the flow cell in the housing when the cover is closed. The system can also contain a plurality of valves that can be individually or collectively closed and opened in order to effect loading of the flow cell. Each of the valves can be individually controlled by a button or lever associated therewith.

A method of loading a sample includes inserting the flow cell into the sample loading apparatus, placing a sample in at least one of the wells of the loading block, activating a vacuum source fluidly coupled to the outlet ports to pull the sample into the channel, and optionally aspirating the unused sample from the well of the loading block.

In one embodiment, a two-sided multi-channel flow cell receives multiple analyte samples in a manner that prevents contamination between the samples. The flow cell has multiple independently-addressable sample channels and uses removable loading blocks for loading sample. The flow cell can form a well for housing an index-matched fluid for observation and analysis of analytes within the multiple flow channels. Each of the flow channels has an inlet and an outlet which mate, respectively, with the loading block and an outlet block and which are also accessible for introduction of chemical reagents after the loading and outlet blocks are removed.

Embodiments of flow cells according to the invention allow for reduced cross-contamination in sample loading and the ability to observe activity within the flow cell once the channels are loaded. They also are useful for chemical analysis in which optical detection is required.

One embodiment of a flow cell according to the invention comprises a plurality of independently-addressable channels sandwiched between a first layer of glass (or other material(s)) and a second layer of glass (or other material(s)). Each of the channels can be coated with a layer that facilitates support-binding of an analyte. The coating can be an epoxide, for example. Each of the channels terminates on one end in an inlet and on the other end in an outlet. A loading block having inlet ports that match the inlets of the channels can be mated to the inlets of the channels, and an outlet block can be mated to the outlets of the channels. Analytes can be introduced into the channels via the inlet ports of the loading block and are pulled through the channels by capillary action or by vacuum. Once analyte has been introduced into each of the channels, the loading and outlet blocks can be removed and the device turned over. A fluid that has an index of refraction that matches the index of refraction of the optical apparatus used to observe the channels can then be introduced into a well defined by the cell. Reagents can then be introduced into each of the channels for chemical reactions therein, excess reagent being washed out through the channel outlets. Observation of optically-detectable moieties is then conducted. With such a flow cell, and with any flow cell according to the invention, optical labels associated with incorporation in a sequencing-by-synthesis reaction can be observed.

If an epoxide coating is used in each of the channels, an amino-terminated nucleic acid primer introduced into the channels will covalently bond to the epoxide coating. Any unbound primer can be washed out of the channels. If the flow cell can then be turned over and the index matched oil placed in the well. Mating the cell with a chuck mounted on an optical microscope allows sample nucleic acid to be introduced through the channel inlets. The introduced nucleic acid hybridizes to the primers. Reagents can then be introduced for sequencing-by-synthesis of the sample (template) nucleic acid in the channels. Sequencing takes place by introducing a nucleotide analog comprising an optically-detectable label and a polymerase into the channels. Template-dependent nucleotide addition occurs in support-bound duplex having an available complementary base. Unbound nucleotide is washed out of the channels and further cycles of base addition are conducted with optical identification of label attached to incorporated nucleotides after the channels are flushed. Labels can be removed or can be eliminated by, for example, photobleaching. Sequence is compiled based upon the linear order of bases incorporated into each duplex. Preferably, duplex are individually optically resolvable.

### BRIEF DESCRIPTION OF THE DRAWINGS

For a fuller understanding of the nature and operation of various embodiments according to the present invention, reference is made to the following description taken in conjunction with the accompanying drawing figures which are not necessarily to scale and wherein like reference characters denote corresponding or related parts throughout the several views and wherein:

FIG. 1 is a schematic diagram of a flow cell of the present invention;

FIG. 2 is a schematic of flow cell coordinate system;

FIG. 3 is a schematic diagram of two imaging areas, A and B, each with three spots, where the two imaging areas are separate flow cells attached to the same holder;

FIG. 4 is a schematic diagram of two imaging areas, A and B, where the surface of a single flow cell is divided into separate areas that have separate fluidic connections;

FIG. 5A shows a schematic diagram of an embodiment of a flow cell that has multiple channels;

FIG. 5B shows a schematic of parallel hydrophobic (hatched) and hydrophilic (open) channels;

FIG. 5C shows a schematic of a flow cell having channels created by hydrophobic and hydrophilic regions;

FIG. 6 depicts an approximation of spots in circular annuli;

FIG. 7 is a schematic top view of an exemplary embodiment of a flow cell;

FIG. 8A is an exploded perspective view of a first embodiment of the flow cell of FIG. 7 showing top and side surfaces of the components that comprise this first embodiment of the flow cell;

FIG. 8B is an exploded perspective view of the of the flow cell of FIG. 8A but showing bottom and side surfaces of the components;

FIG. 8C is a perspective view like FIG. 8B but with two substrates assembled;

FIG. 9A is an exploded perspective view of a second embodiment of the flow cell of FIG. 7 showing top and side surfaces of the components that comprise this second embodiment of the flow cell;

FIG. 9B is an exploded perspective of the flow cell of FIG. 9A but showing bottom and side surfaces of the components;

FIG. 9C is a perspective view like FIG. 9B but with the adhesive film disposed on the first substrate;

FIG. 9D is a perspective view of a fully assembled flow cell of FIGS. 9A-9C;

FIG. 10A is a perspective view according to an exemplary embodiment of a fully assembled flow cell;

FIG. 10B is a perspective view of the flow cell of FIG. 10A with a loading block being moved into position;

FIG. 10C is a perspective bottom view of the flow cell of FIG. 10B;

FIG. 10D is a perspective view according to an exemplary embodiment of a loading block;

FIG. 10E is a perspective view of the flow cell of FIG. 10B after the loading block has been moved into position;

FIG. 10F is a perspective view of the flow cell of FIG. 10E with an unloading block being moved into position;

FIG. 10G is a perspective bottom view of the flow cell of FIG. 10F;

FIG. 10H is a perspective view according to an exemplary embodiment of the flow cell shown in FIG. 10F after the unloading block has been moved into position;

FIG. 11A is a schematic view of a sample loading apparatus according to one exemplary embodiment of the present invention;

FIG. 11B is a partially cut-away view of the sample loading apparatus shown in FIG. 11A;

FIG. 11C is an exploded view of the sample loading apparatus shown in FIG. 11A;

FIG. 11D is a side view of a sample loading apparatus;

FIG. 11E is a schematic of the microfluidic handling system, temperature control system, and the vacuum system of a sample loading apparatus;

FIG. 11F is a schematic view of the sample loading apparatus shown in FIG. 11A with a flow cell loaded into position;

FIG. 11G is a schematic view of the microfluidic handling system of a sample loading apparatus;

FIG. 11H is a schematic view of the sample loading apparatus shown in FIG. 11A with the cover in a closed position;

FIG. 11 is a side view of the sample loading apparatus shown in FIG. 11D with the cover in a closed position;

FIG. 11J is an enlarged view of the latch shown in FIG. 11I;

FIG. 12 is a schematic view of an apparatus that can be used to perform analytical experimentation with an exemplary embodiment of the flow cell shown in FIG. 10A;

FIG. 13A is a perspective view of an inverted flow cell being placed into a flow chuck;

FIG. 13B is a perspective bottom view of the flow cell shown in FIG. 13A;

FIG. 13C is a perspective view of the flow cell shown in FIG. 13A after it has been placed in the flow chuck;

FIG. 14A is a perspective view of an inverted flow cell being placed into a flow chuck;

FIG. 14B is a perspective bottom view of the flow cell shown in FIG. 14A;

FIG. 14C is a perspective view of the flow cell shown in FIG. 14A after it has been placed in the flow chuck;

FIG. 15 is a perspective view of a dual flow cell assembly;

FIG. 16 is a schematic of an imaging area having multiple spots of biochemical molecules attached thereto;

FIG. 17 is an exemplary schematic showing molecules viewed as an image stack;

FIG. 18 shows an exemplary imaging system of the present invention;

FIG. 19A is a chart showing an estimation of the number of targets per flow cell versus sequence length of the target nucleic acid of interest; and

FIG. 19B is a chart showing an estimation of the spot diameter versus kilobases of target nucleic acid of interest.

### DESCRIPTION

Embodiments of fluidic apparatus according to the invention generally streamline the analysis of biochemical assays. Each of the embodiments enables reaction and interrogation components of biochemical reactions to occur in parallel, thereby reducing total cycle time for a biochemical assay. With certain embodiments, the total cycle time for a biochemical assay is reduced to about the time needed to interrogate a compartment containing reaction products. Where the biochemical assay is performed one or more times on the same flow cell according to the invention, a significant time savings is realized as is full utilization of the cell, with little downtime during execution of the reaction component (also referred to herein as reaction time) of the biochemical assay.

Embodiments of flow cells according to the invention are designed to allow parallel processing of biochemical assays that have a reaction component and an interrogation component, where the reaction and interrogation components are typically performed in sequence in (or on) the same flow cell. Generally, the flow cells have two or more compartments that have independent and separate plumbing such that the reaction can be conducted in one compartment while imaging is separately taking place in another compartment. Flow cell compartments (or subcompartments) can be fluidly isolated from each other such that they are not in fluid communication with each other.

In a first exemplary embodiment according to the invention, an apparatus comprises two or more multi-channel flow cells attached to the same holder. Each of the attached flow cells comprises one or more imaging areas and has separate fluidic connections, such that the reaction component of the biochemical assay can be performed on one flow cell without affecting the imaging areas of the other flow cells.

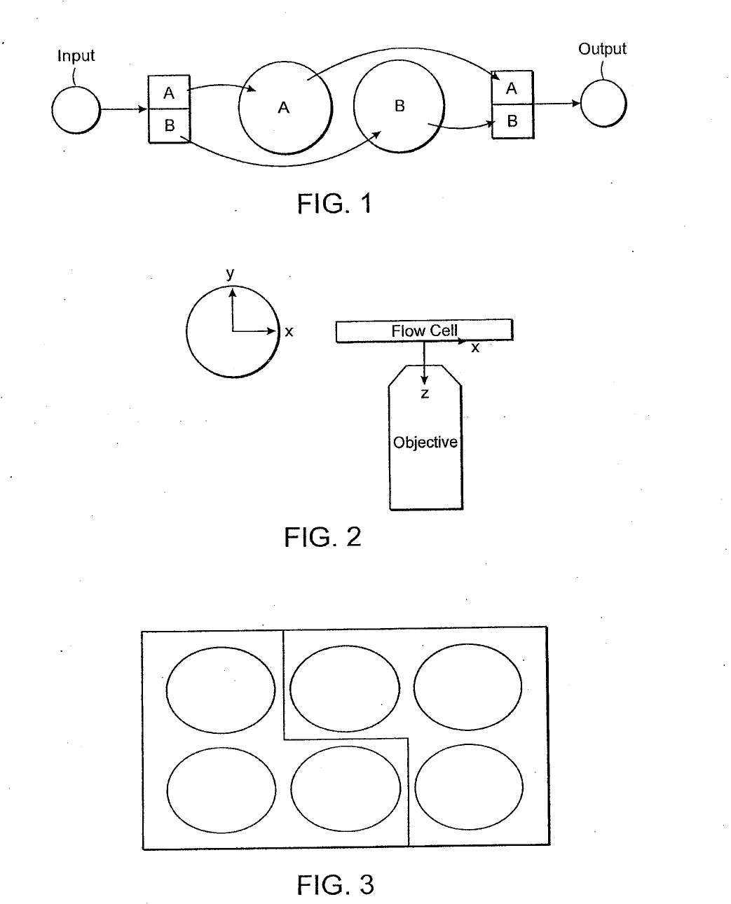

In general, embodiments of flow cells according to the invention can be made using any appropriate surface as described herein. One surface for the imaging areas of a flow cells is an epoxide surface on a glass or fused silica slide or cover slip. For example, the surface can be about a 10 mm to about a 100 mm round cover glass. The surface can have a thickness of about 0.05 mm to about 0.45 mm. In one embodiment, the cover glass is a 40 mm round cover glass (Erie Scientific) and has a thickness of 0.15 mm. The imagable area of the cover glass can be from about 10 mm² to about 10,000 mm². In one embodiment, the imagable area of the cover glass is about 690 mm², which is split amongst the imaging areas of the flow cell. Where the flow cell comprises two imaging areas, each imaging area can be about 345 mm². FIG. 1 is a schematic diagram of an exemplary flow cell.

Each imaging area of the flow cell can have independent microfluidic plumbing. In other words, each imaging area can have its own fluid inlet port and/or fluid outlet port. In addition, each imaging area can be thermally and hydraulically independent, thereby allowing the reaction component to be performed on one imaging area while another imaging area is interrogated (e.g., imaged). The cover glass can include a guard band of about 2 mm, or the edges of the cover glass can be sloped so that the interrogation device (e.g., a Nikon Plan APO TIRF 60x/1.45 objective does not interfere with the imaging areas. Referring to the particular embodiment of FIG. 2, the X-Y tilt of the objective is about 1 nm/μm (1 milliradian) and the flatness is 100 nm peak to peak in 100 μm. The Z placement repeatability of the objective (e.g., mount and dismount from the flow cell is 10 μm +/−2 standard deviations. The parking area for the objective lens is thermally isolated from the flow cell, and there is a re-oiling area for the objective lens.

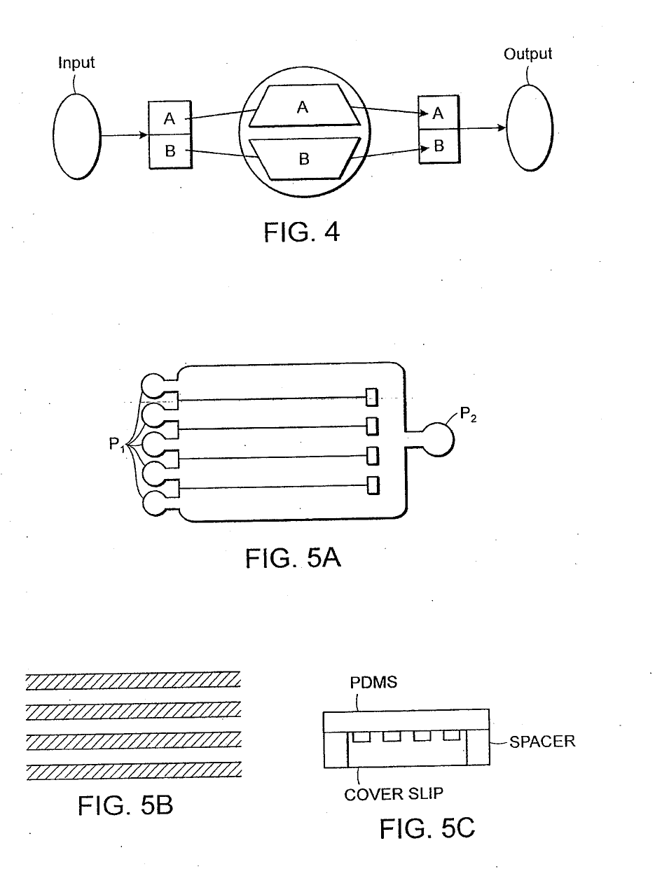

As described above, the imaging areas can be part of separate flow cells that are attached to the same holder and mounted together into the interrogation device (such as a microscope). In another embodiment, the imaging areas are part of a single flow cell and each imaging area is surrounded by a gasket that isolates the imaging areas from each other as described above. FIG. 3 is a schematic diagram of two imaging areas, A and B, each with three spots, where the two imaging areas are separate flow cells attached to the same holder. FIG. 4 is a schematic diagram of two imaging areas, A and B, where the surface of a single flow cell is divided into separate areas that have separate fluidic connections.

As shown in FIG. 5A, one embodiment of a flow cell can comprise a series of two or more flow paths (also referred to herein as channels). Each channel can have a separate fluid inlet P₁. The channels can have separate fluid outlets or can share a common fluid outlet P₂. The channels can be formed by masking. For example, parallel hydrophobic and hydrophilic regions can be created, as shown in FIG. 5B. Hydrophobic and hydrophilic regions can be formed by using a glass cover slip and a polydimethylsiloxane (PDMS) slide as shown in FIG. 5C. The PDMS surface can be made selectively hydrophobic by masking and exposing to plasma.

The reagents required for performing the reaction component of the biochemical assay are flowed in simultaneously. Because the flows in each channel usually have very low Reynolds Numbers (<<1), and typically have the same viscosity, applying constant pressure down the channels results in multiple parallel flow paths. The application of pressure down the channels can be accomplished by applying pressure to the inlet, applying suction to the outlet, or a combination of both to achieve a suitable flow rate through the channels. Therefore, where the biochemical assay involves nucleic acids, nucleic acids can be attached to the channels as described above. Multiple different oligonucleotides can then be added to the channels and hybridized to the attached nucleic acids in one step. In addition, depending on the ratio of the imaging time compared to the reaction time as described above, the channels can be divided into two or more groups such that one group of channels is subjected to the reaction component of the biochemical assay, while the other group of channels is interrogated.

The cover glass can include a guard band of about 2 mm, or the edges of the cover glass can be sloped so that the interrogation device does not interfere with the imaging areas. In one embodiment, the interrogation device is a Nikon Plan APO TIRF 60x/1.45 objective. Other suitable interrogation or detection devices are described below. Referring to FIG. 2, the X-Y tilt of the objective can be about 0.1 to about 10 milliradians. In one embodiment, the X-Y tilt of is about 1 nm/μm (1 milliradian). In one embodiment, the Z placement repeatability of the objective (e.g., mount and dismount from the flow cell) is about 10 μm +1-2 standard deviations. The parking area for the objective lens can be thermally isolated from the flow cell.

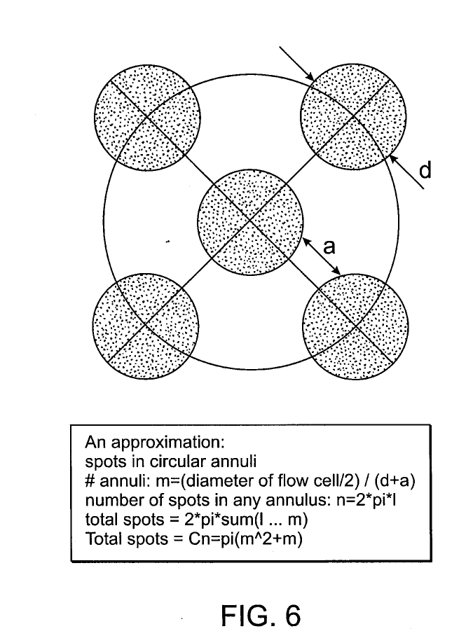

As described herein, flow cells having minimal volume provide several advantages. For example, the volume of the flow cell can be from about 1 to about 1000 μl. Furthermore, the exchange of internal volume of the flow cells is rapid. In one embodiment, the exchange takes less than 1 second at 3000 kPa driving pressure and the maximum Reynolds Number at 4° C. is less than 1. The bow of the cover slip is typically less than 20% of initial channel height during pumping. FIG. 6 depicts an approximation of spots in circular annuli.

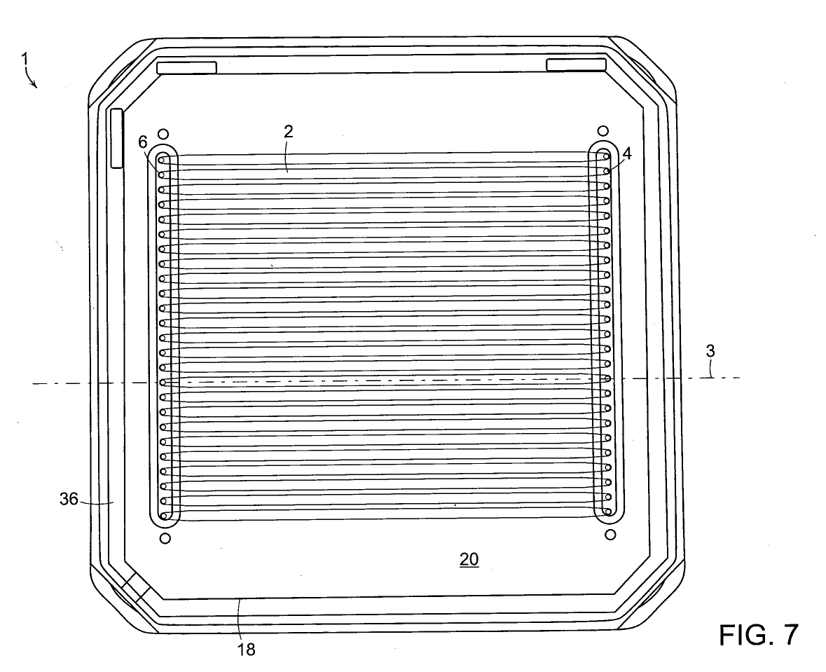

A second exemplary embodiment of a multi-channel flow cell for handling and analyzing microfluidic volumes and related biological materials is designated 1 in FIG. 7. The multi-channel flow cell can be used in a wide variety of applications such as, for example, performing single molecule sequencing.

Referring now to FIG. 7, the flow cell 1 includes a plurality of channels 2 oriented parallel to each other. Each channel 2 has an inlet 4 for loading a fluid into the channel 2, and an outlet 6 for removing fluid from the channel 2. The channels 2 each have a capacity of about 3 microliters to about 15 microliters. Each of the channels 2 extends longitudinally along an axis 3 from one of the inlets 4 to a corresponding one of the outlets 6. As shown, the channels 2 have a uniform width throughout the axis 3, however they may be tapered or curved in width and/or in depth depending on the desired application of the flow cell 1. Also, multiple channels 2 are shown but of course the flow cell 1 can have just a single one of the channels 2.

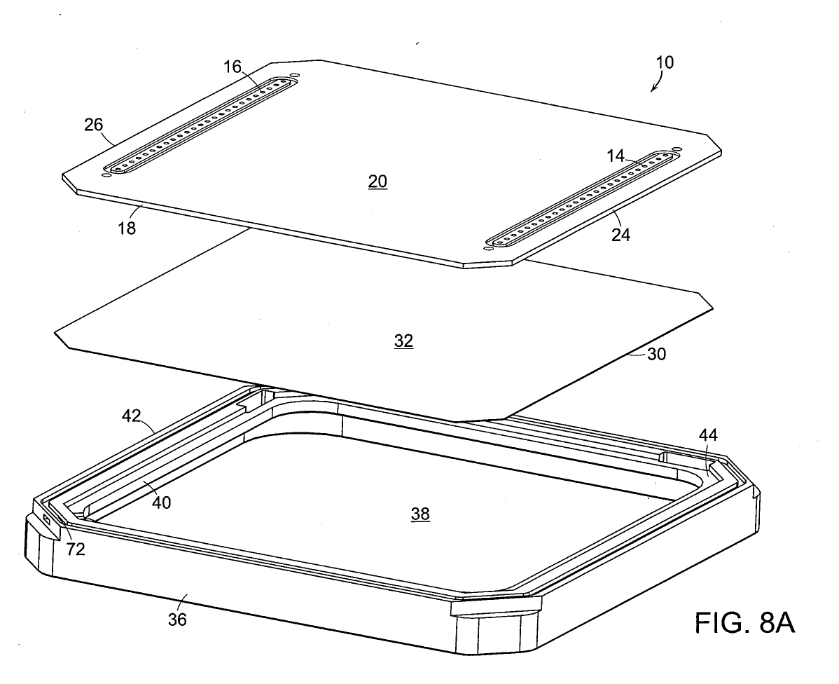

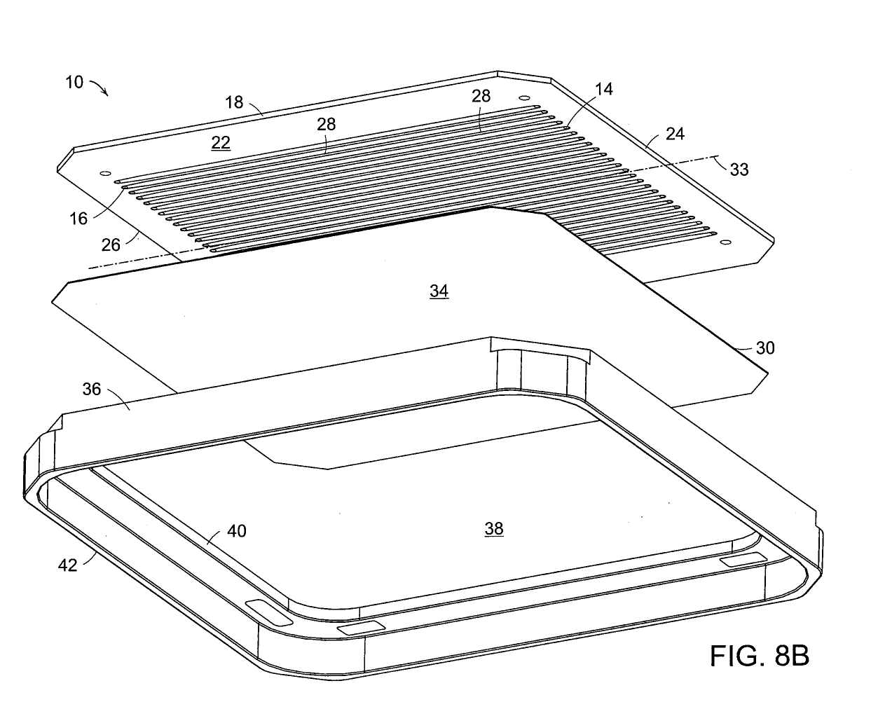

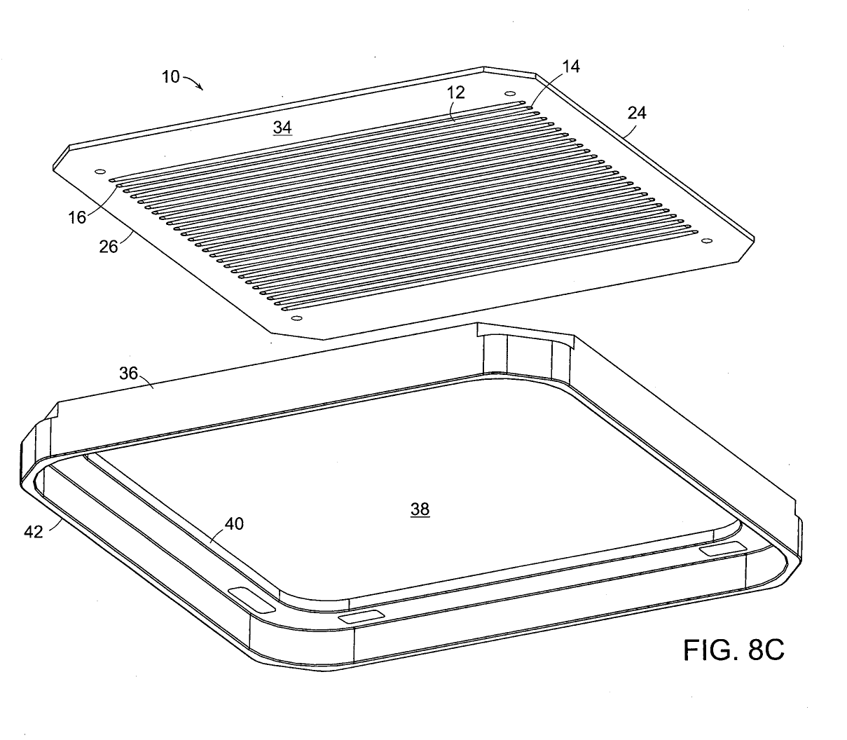

Referring now to FIGS. 8A-8C, individual components of a first embodiment of a flow cell 10 are shown prior to assembly. This flow cell 10 includes a first substrate 18 having a top surface 20 (FIG. 8A) and a bottom surface 22 (FIG. 8B). The first substrate 18 further includes a plurality of inlet holes 14 and a plurality of outlet holes 16 formed therein, and each of these holes 14, 16 extends through the substrate from the top surface 20 of the first substrate 18 to its bottom surface 22. The inlet holes 14 are aligned in a row near one edge 24 of the first substrate 18, and the outlet holes 16 are aligned in a row near an opposing edge 26 of the first substrate 18.

As best shown in FIG. 8B, each inlet hole 14 has an corresponding or matching outlet hole 16. A plurality of recesses 28 are etched, carved or molded into the bottom surface 22 of the first substrate 18 along an axis 33 extending from each inlet hole 14 to its corresponding outlet hole 16. The first substrate 18 may be formed to any desired depth or width needed to form the desired channel 12 size and shape. In one embodiment, the size of the channels is chosen to make sure that the flow remains laminar at the desired flow rate.

The flow cell 10 further includes a second substrate 30 having a top surface 32 (FIG. 8A) and a bottom surface 34 (FIG. 8B). The second substrate 30 is for assembly to the first substrate 18, such that the top surface 32 of the second substrate 30 contacts the bottom surface 20 of the first substrate 18. Once the two substrates 18, 30 have been secured to each other, the recesses 28 are sealed and form channels 12 (FIG. 8C). In alternative embodiments, the recesses 28 can be etched, carved or molded into the top surface 32 of the second substrate 30. The first substrate 18 can be selectively attached to the second substrate 30 by use of a variety of mechanical fasteners or by use of any of a variety of adhesives applied to the top surface 32 and/or the bottom surface 22.

The first substrate 18 and the second substrate 30 each can be manufactured from any of a variety of materials or combinations of materials as long as the substrates 18, are compatible with the microliter volumes passed therethrough, and to which any substances in the microliter volumes will not stick. The surfaces 22, 32 can also be passivated so that samples, such as DNA, only adhere to the desired surface. Passivation reagents include, for example, amines, phosphate, water, sulfates, detergents, bovine serum albumin (BSA), human serum albumin (HSA) or polymers such as POP-6® sold by Applied Biosystems.

The substrates 18, 30 are generally formed of a material that will allow light and/or energy of appropriate wavelength(s) to pass therethrough. This is because light of one or more wavelengths is passed through the substrates 18, 30 to illuminate the material(s) within the channels 12, in one use of the flow cell 10. The substrates 18, 30 can be glass, fused silica, sapphire, polydimethylsiloxane (PDMS), polymethyl methacrylate (PMMA) or a suitable clear plastic such as acrylic or polycarbonate. In some embodiments, the materials used as substrates 18, 30 can be the same or different, such that adhesives and/or mechanical fasteners are not necessary to secure the substrates 18, 30 to each other.

The flow cell 10 further includes a frame 36. The frame 36 includes an inside edge 40 and an outside edge 42, and the frame 36 defines a rectangular shaped opening 38. The frame 36 further includes a recess 44 (FIG. 8A) formed along the inside edge 40 for receiving and selectively securing (with, for example, an adhesive) the assembled substrates 18, 30 to the frame 36. The frame 36 can be constructed of any material with thermal expansion characteristics similar to those of the two substrates 18, 30, such as, for example, glass filled polycarbonate or multiple composite plastic. Alternatively, if there is a thermal mis-match, a highly-flexible glue, such as silicone, can be used.

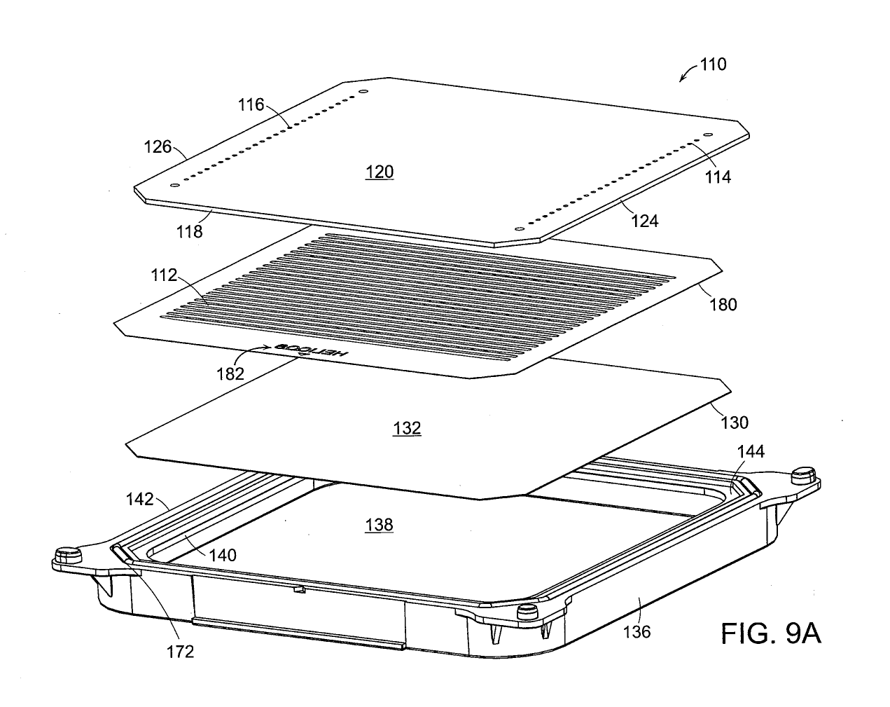

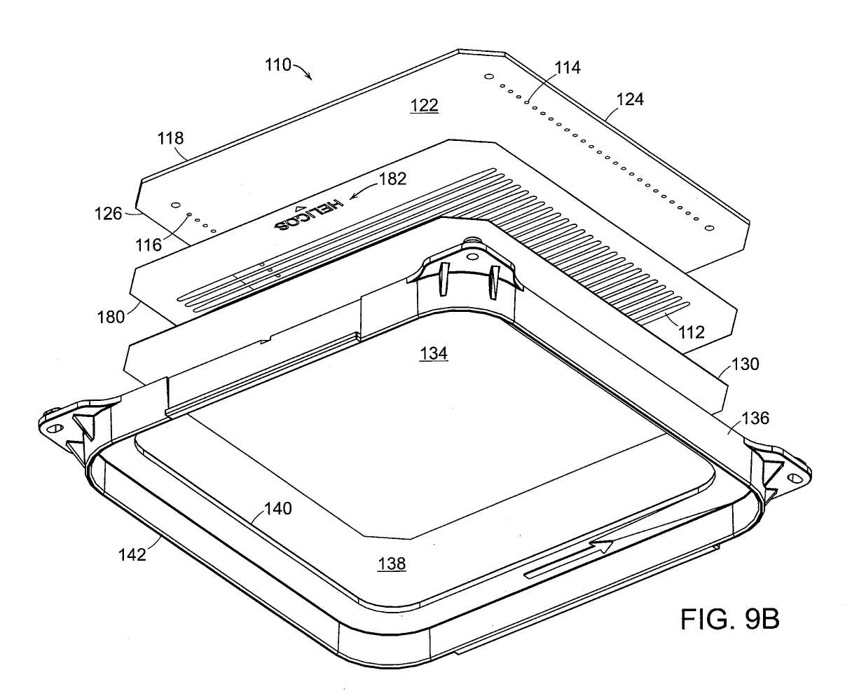

Turning to FIGS. 9A-9D, a third exemplary embodiment of a flow cell 110 according to the invention includes channels 112 formed in a manner different than described above with reference to FIGS. 8A-8C.

An adhesive can be disposed on a film in a desired bonding pattern. The film can then be used to apply the patterned adhesive to one surface of a material to be bonded and then the film is pealed away and discarded leaving behind the adhesive disposed on the surface only in the desired locations and in the desired pattern. A second material can then be placed in contact with the first material to bond the two materials. Micronics, Inc. of Redmond, Wash. is one supplier of this technology.

The flow cell 110 includes a first substrate 118 having a top surface 120 and a bottom surface 122. The first substrate 118 further includes a plurality of inlet holes 114 and a plurality of outlet holes 116. A pattern of adhesive is disposed on a piece of film 180 in a predetermined bonding pattern. Using the technique described above, the film 180 is used to apply the adhesive pattern to the bottom surface 122 of the first substrate 118 (FIG. 9C).

The flow cell 110 further includes a second substrate 130 having a top surface 132 and a bottom surface 134. As an alternative to applying the adhesive to the bottom surface 122 of the first substrate 118 as described above, the adhesive may be disposed on the top surface 132 of the second substrate 130. Once the adhesive is applied to one of the surfaces 122, 132, the two substrates 118, 130 are aligned and placed in contact with each other. After the adhesive cures, the two substrates 118, 130 are bonded together, and the channels 112 are formed in the regions where no adhesive was disposed on either substrate 118, 130. The layer of adhesive has any predetermined thickness and pattern in order to create channels 112 with desired dimensions and shapes. Furthermore, additional patterns and reference features, such as, arrows, logos 182 or written instructions can also be included in the adhesive layer to better ensure proper operation by the user.

Referring now to FIG. 9A, the flow cell 110 further includes a frame 136. The frame 136 has an inside edge 140 and an outside edge 142, and the frame 136 defines a rectangular shaped opening 138. The frame 136 includes a recess 144 formed along the inside edge 140 for receiving and selectively securing (with, for example, an adhesive) the substrates 118, 130 to the frame 136. The frame 136 also includes a recess 172 formed around the outside perimeter near the outside edge 142. This recess 172 can optionally receive a gasket or compressible tubing to improve the seal when the flow cell 110 is being used in operation.

FIG. 9D shows a fully assembled flow cell 110 ready to be loaded by a user. As shown, the flow cell 110 has gaskets 115, 117 in place surrounding the inlet holes 114 and outlet holes 116. The gaskets 115, 117 can be placed in recesses or can be placed on a flat top surface 120 depending on the desired application. The flow cell 110 also has a compressible tube 190 disposed in the recess 172.

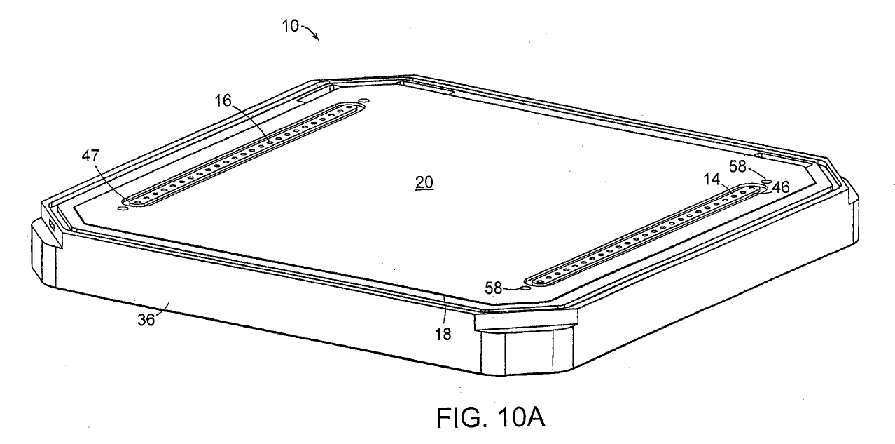

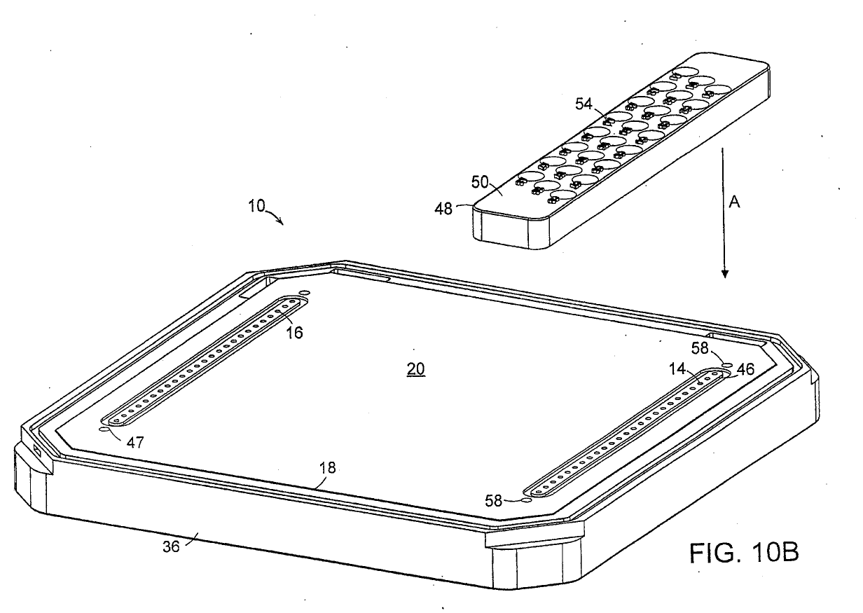

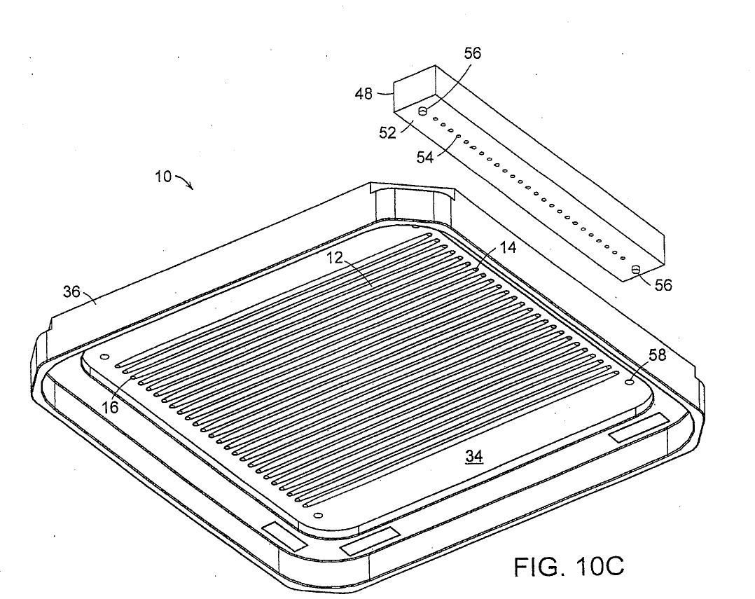

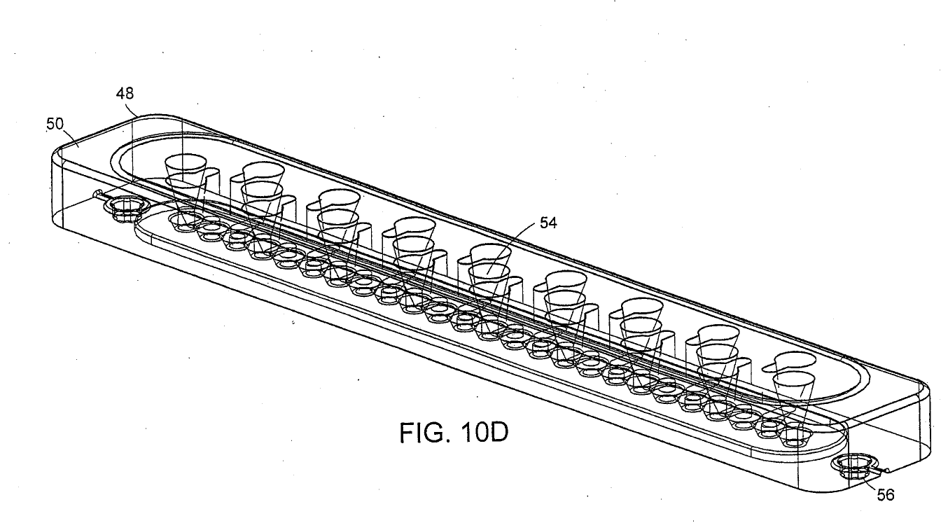

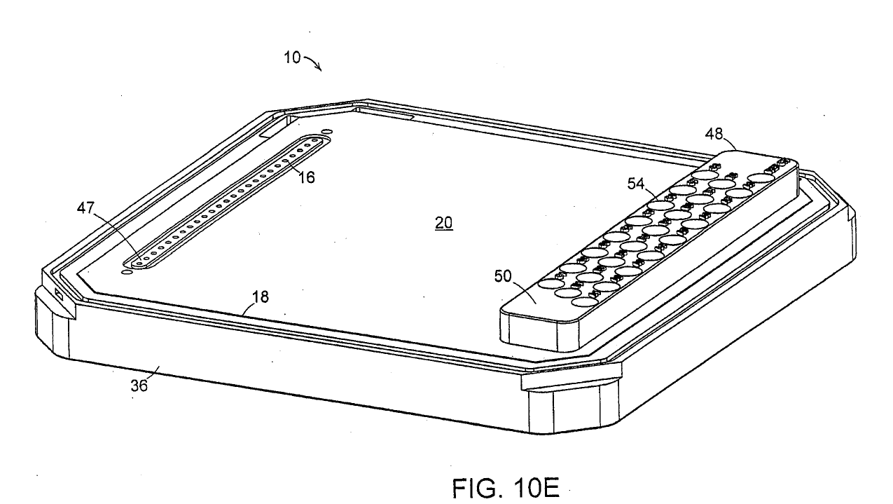

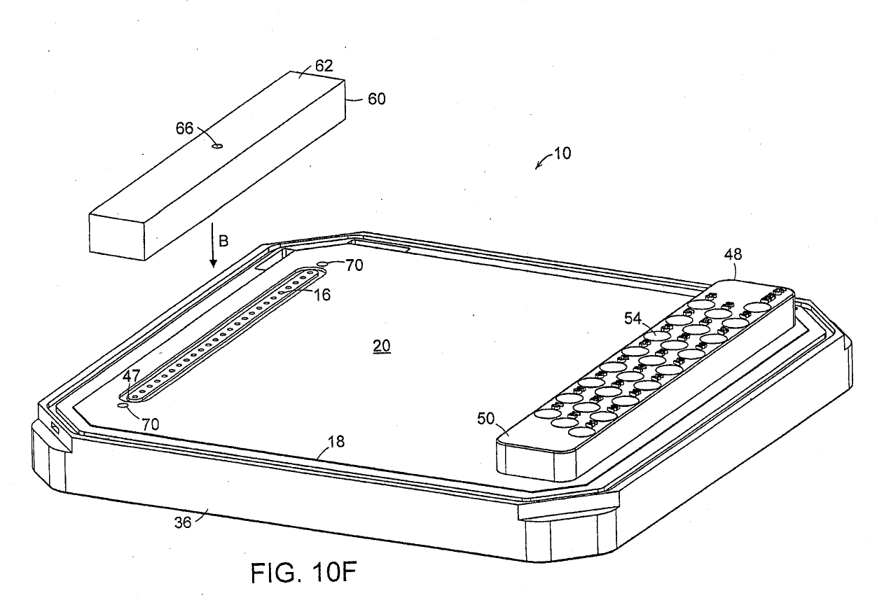

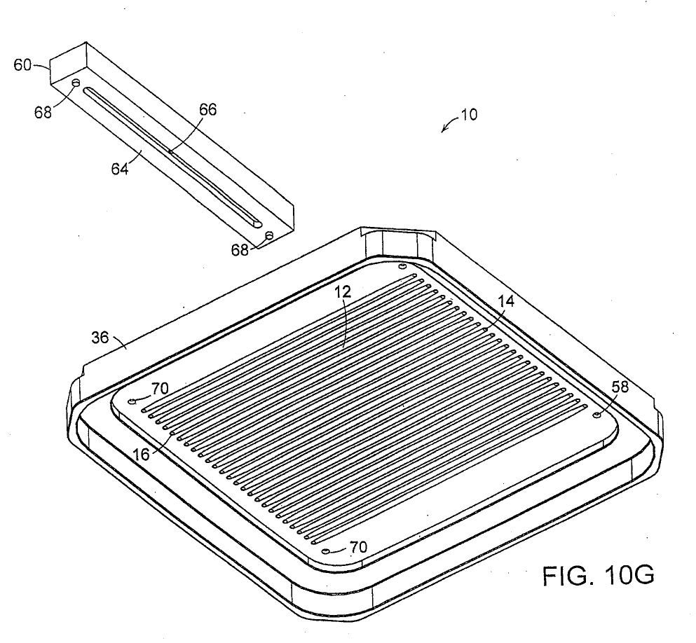

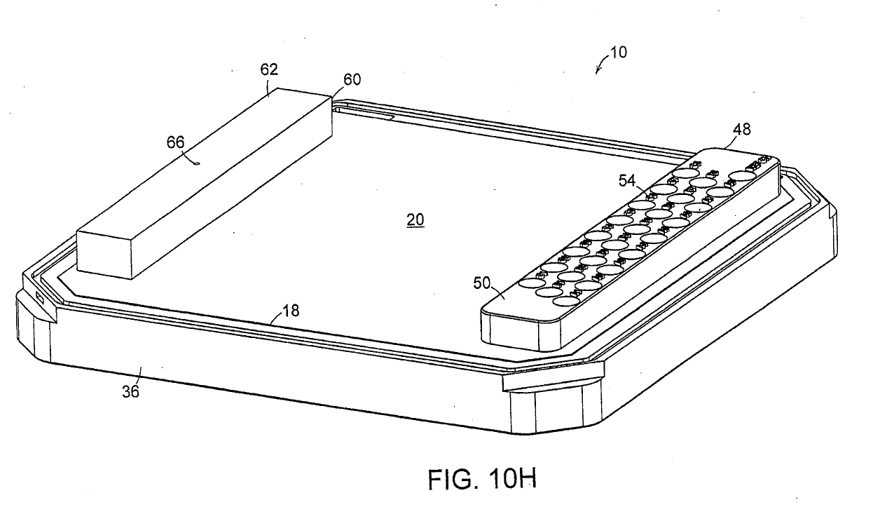

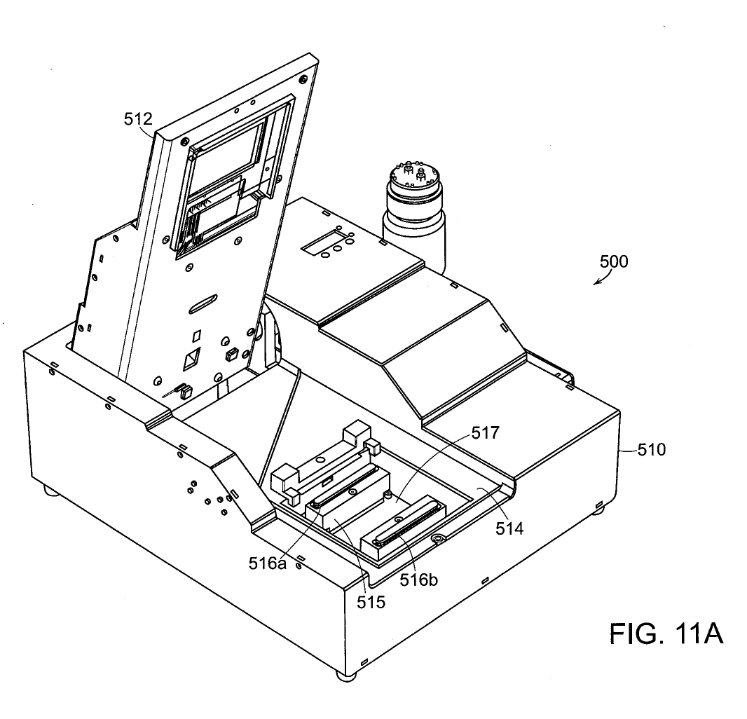

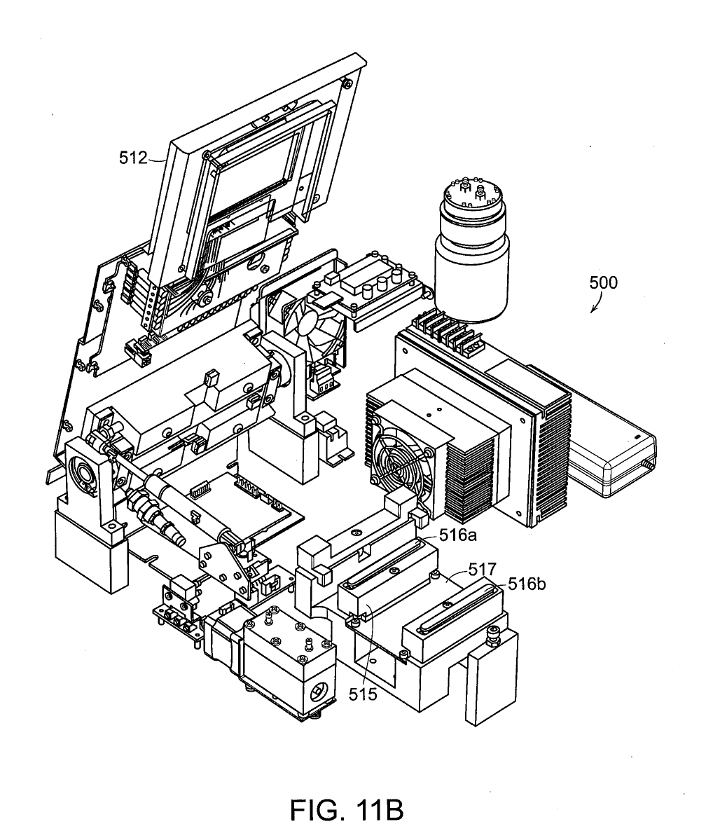

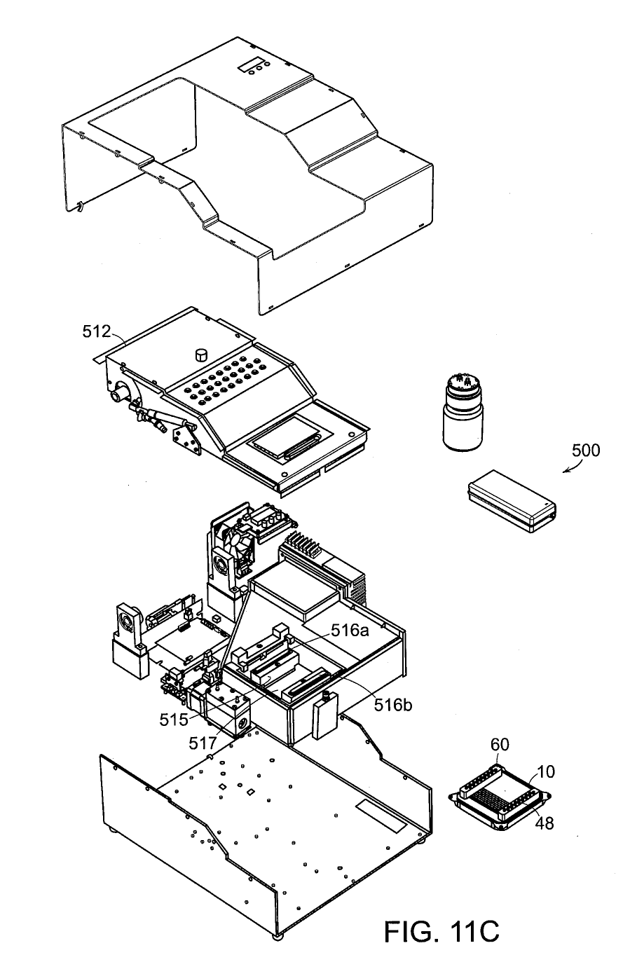

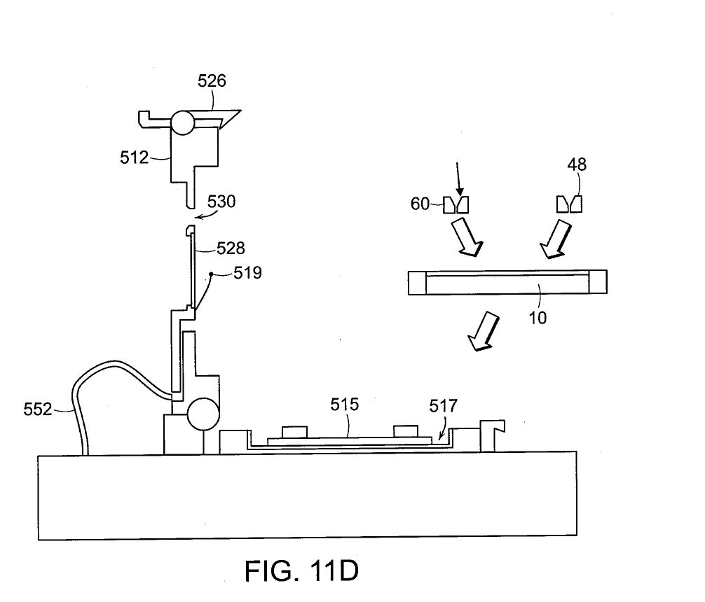

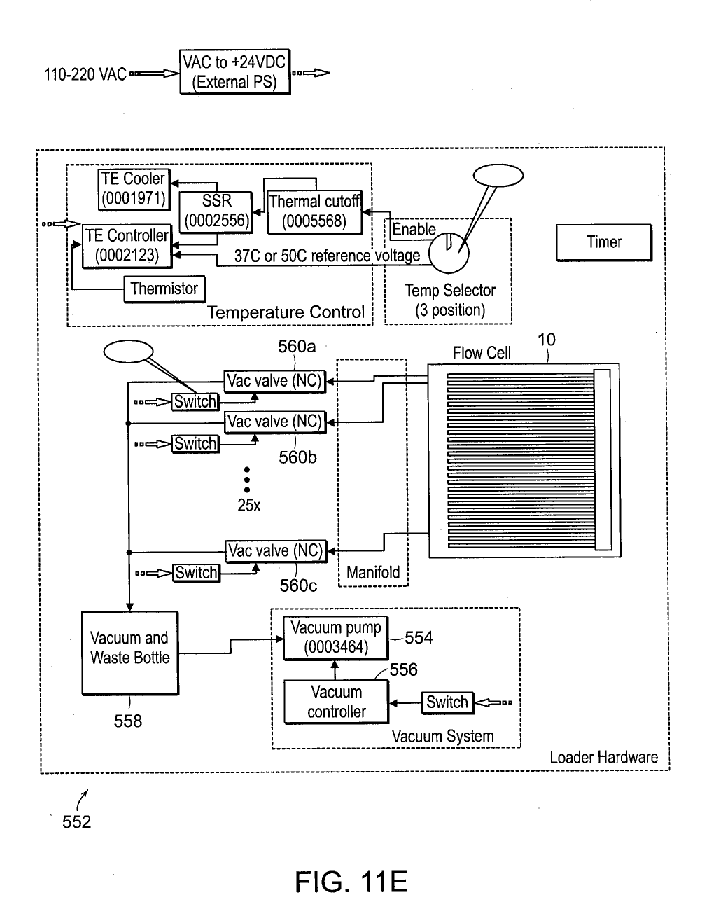

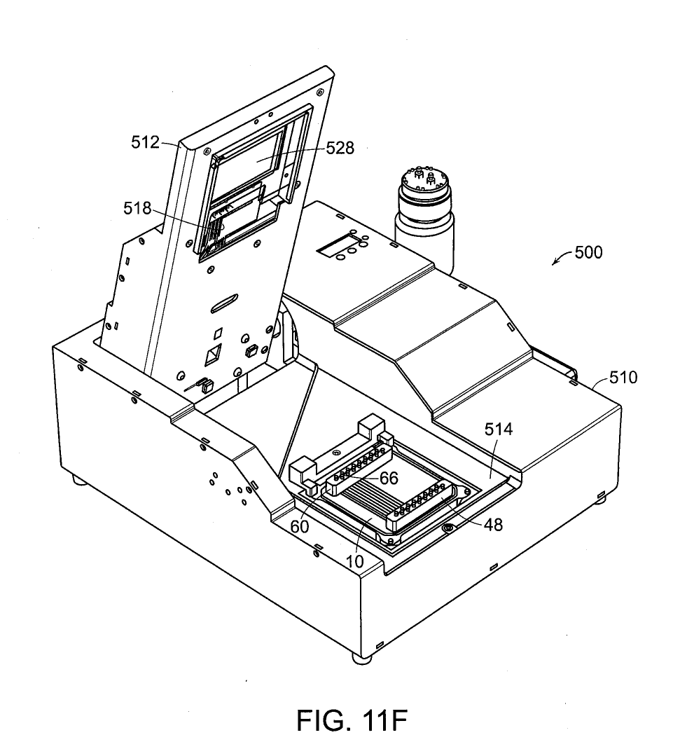

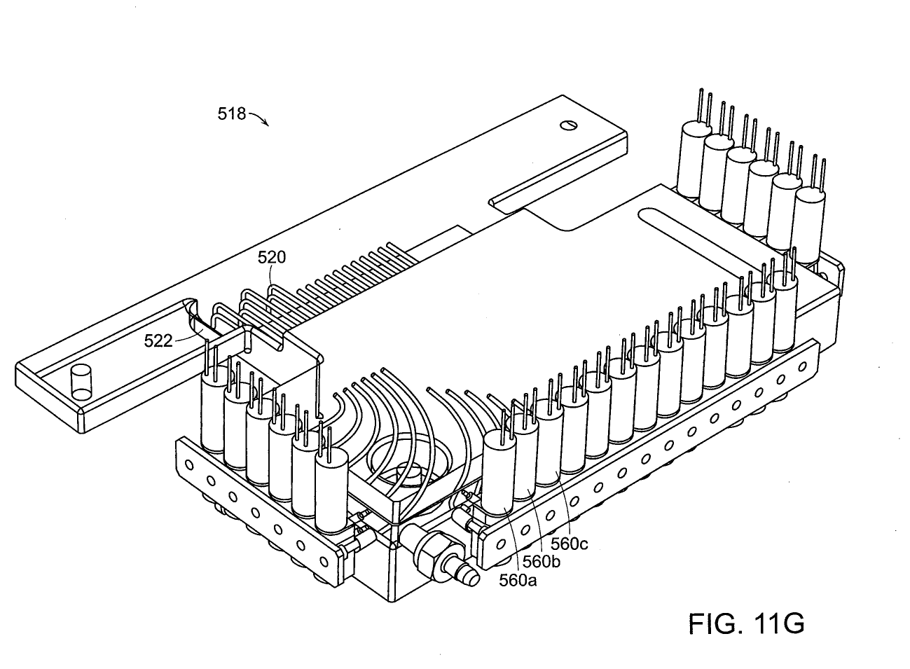

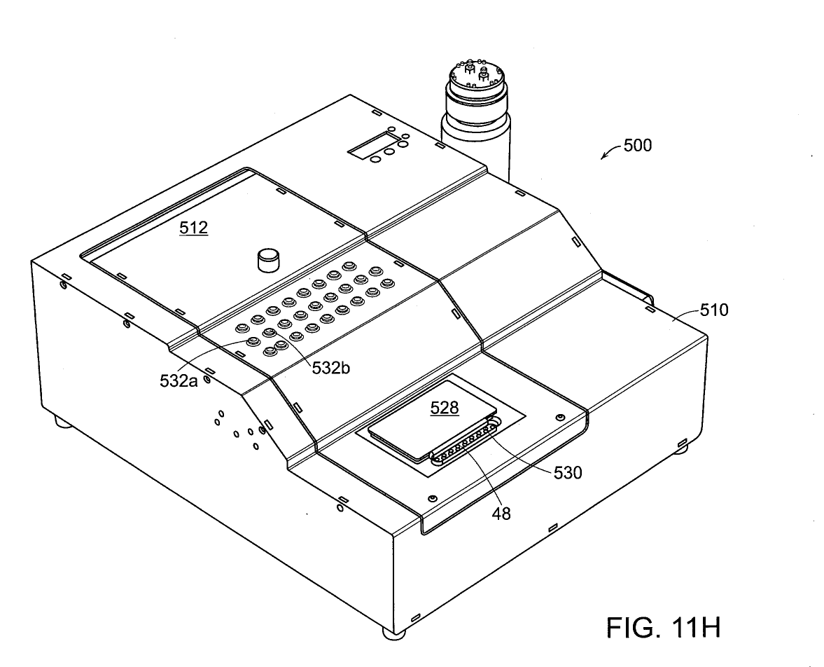

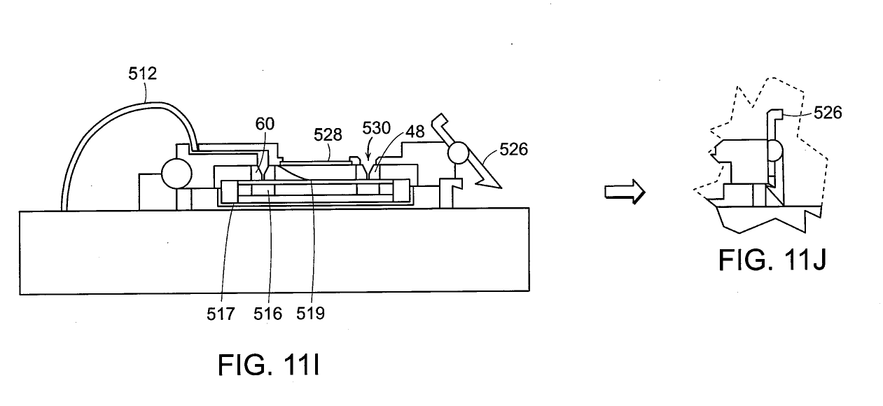

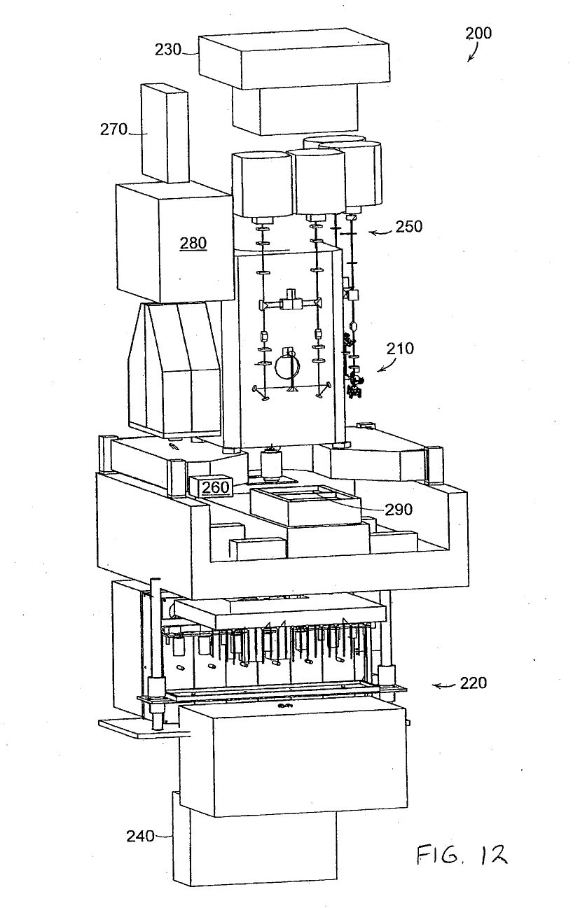

Turning now to FIGS. 10A-11J, wherein the process of loading samples into the flow cell 10 is shown and described. Referring now to FIG. 10A, there is shown a fully assembled flow cell 10. Although not shown in this particular illustration, each inlet hole 14 is coupled to a channel 12, which is coupled to an outlet hole 16, such that when a fluid is loaded into the inlet hole 14, it can flow through a channel 12 and then be removed via the outlet hole 16. Optionally, a recessed canal 46 is formed in the top surface 20 of the first substrate 18 completely surrounding the inlet holes 14. An additional recessed canal 47 is formed in the top surface 20 of the first substrate 18 completely surrounding the outlet holes 16. These canals 46, 47 can optionally receive a gasket or compressible tubing for sealing the inlet holes 14 and outlet holes 16 during the processes of loading and unloading of the channels 12 and when the flow cell 10 is being used in operation.

Referring now to FIGS. 10B-10E, a loading block 48 is provided for loading fluids into the channels 12 of the flow cell 10 for analysis. The loading block 48 has a top surface 50 and a bottom surface 52. The loading block 48 includes a plurality of loading wells 54 that extend through the loading block 48 from the top surface 50 to the bottom surface 52. As shown, the loading wells 54 are essentially conically shaped with the widest diameter near the top surface 50 and the narrowest diameter near the bottom surface 52. At the top surface 50, the loading wells 54 are arranged in three staggered rows. The loading wells 54 then angle toward the center of the loading block 48 forming a single row (FIG. 10C) at the bottom surface 52, such that the loading wells 54 align with the inlet holes 14. Alternatively, as shown in FIG. 10D, the loading wells 54 in the center row can be circular at the top surface 50 and the two outside rows can have a keyhole shape at the top surface 50. The loading wells 54 then taper down to a single row at the bottom surface 52 of the loading block 48. The loading wells 54 can be any shape or size necessary to facilitate loading a sufficient amount of sample into the channels 12 of the flow cell 10.

The loading block 48 further includes two raised features knows as mating pins 56 protruding from the bottom surface 52. The mating pins 56 align with, and are received into receive holes 58 in the top surface 20 of the first substrate 18. When the loading block 48 is lowered onto the flow cell 10 in the direction indicated by line A on FIG. 10B, the mating pins 56 are inserted into the receiving holes 58. This ensures proper alignment of the loading wells 54 and the inlet holes 14, such that fluid can flow from the loading wells 54, into the inlet hole 14 and then into the channel 12. A gasket or compressible tubing (not shown) may be installed in the canal 46 to provide a tight seal around the inlet holes 14 so that during the loading process, the fluid is contained in the loading block 48 and channels 12 and does not leak onto other areas of the flow cell 10. Alternatively, the loading block 48 can be made from a relatively soft, pliable material (e.g., silicone rubber) with additional raised features on the bottom surface 52 around each loading well 54 so that the loading block 48 itself forms the tight seal without a gasket. The additional raised features on the loading block 48 ensure an effective seal between the loading wells 54 and the top surface 20. These raised features are ridges which can either be rectangular or hemi-circular in cross section in order to provide the correct sealing geometry. FIG. 10E illustrates the loading block 48 in position on the flow cell 10.

Referring now to FIGS. 10F-10H, an unloading block 60 is provided for removing fluids from the channels 12. The unloading block 60 has a top surface 62 and a bottom surface 64. As best shown in FIG. 10F, the unloading block 60 includes a single aperture 66, extending through the unloading block 60 from the top surface 62 to the bottom surface 64. As shown in FIGS. 10F and 10G, the aperture 66 is cylindrical near the top surface 62, and a groove at the bottom surface 64. However, it will we apparent to one skilled in the art that other shapes and sizes of apertures may be formed in the unloading block 60 for removing fluid from the channels 12. Examples include, a plurality of holes or a single duct extending though the unloading block 60. In an alternative embodiment, the block or manifold that connects each channel 12 to its neighbors can be etched or machined directly into the glass of the flow cell. In one embodiment, the unloading block 60 and/or the interface between the unloading block 60 and the outlet hole 16 is designed such that when the vacuum is applied during evacuation of the channels 12 (or while processing samples), there is a uniform pressure distribution across all of the outlet holes 16. The uniform pressure distribution equalized the flow rates of samples and reagents in the channels 12. An optional surface treatment can be added to the channels to make them hydrophobic or hydrophilic in order to control the flow and to prevent it from moving from channel to channel.

The unloading block 60 further includes two raised features knows as mating pins 68 protruding from the bottom surface 64. The mating pins 68 align with, and are received into receive holes 70 in the top surface 20 of the first substrate 18. When the unloading block 60 is lowered onto the flow cell 10 in the direction indicated by line B on FIG. 10F, the mating pins 68 are inserted into the receiving holes 70. This ensures proper alignment of the aperture 66 and the outlet holes 16, such that fluid can flow from the channels 12, through the outlet holes 16 and out of the flow cell 10 through the aperture 66. A gasket or compressible tubing (not shown) installed in the canal 47 provides a tight seal around the inlet holes 14 so that during the unloading process, the fluid is contained in the unloading block 60 and does not leak onto other areas of the flow cell 10. As with the loading block 48, the unloading block 60 can be made from a relatively soft, pliable material (e.g., silicone rubber) with additional raised features on the bottom surface 64 around the aperture 66 so that the unloading block 60 itself forms the tight seal without a gasket. The additional raised features on the unloading block 60 ensure an effective seal between the aperture and the top surface 20. These raised features are ridges which can either be rectangular or hemi-circular in cross section in order to provide the correct sealing geometry. FIG. 10H illustrates the flow cell 10 with the loading block 48 and unloading block 60 in position and ready to for handling microfluidic volumes and related biological materials.

Referring now to FIGS. 11A-11J a sample loading apparatus 500 is shown. The apparatus 500 includes a base 510, a cover 512 pivotally attached to the base 510, and a vacuum system 552. The base 510 defines a chamber 514 for receiving a flow cell. The chamber 514 includes a heater 515 which can be mounted on one or more springs (not shown) that allow at least some compression when the cover 510 is closed. This compression helps to provide a tight seal between the loading/unloading blocks and the flow cell 10. Alternatively, the heater 515 can be mounted on an elastic insulating material 517 that recovers its shape after being compressed to help provide a tight seal and insulate the heater 515 from the rest of the apparatus 500. Supports 516a, 516b (collectively 516) help ensure proper positioning of the flow cell within the chamber 512 and provide support for the pressure exerted on the loading 48 and unloading blocks 60 by the cover 512 when it is closed.

In the embodiment depicted in FIG. 11E, the vacuum system 552 is passive and includes a vacuum pump 554, a drive motor 556, and a reservoir 558. Alternatively, the vacuum system 552 could be non-passive, where the vacuum pump 554 is directly connected to the flow cell. In one embodiment, the vacuum pump 554 is a compact rotary vane type pump; however, the pump size and type will be selected to suit the particular application. For example, the pump could be a piston, gear, or diaphragm type pump. Further, the pump size will depend on the operating parameters of the apparatus 500, for example, the larger the pump capacity, the quicker the pump 554 will evacuate the reservoir 558. The drive motor a 556 in one embodiment is a 12 volt DC electric motor; however, the motor size and type will be selected to suit the particular application. For example, larger flows may require a larger pump, which in turn may require a larger motor. Further, the pump 554 can be uni- or bidirectional and can be coupled to the motor 554 directly or via a flexible coupling or other means known to one of skill in the art. In a particular embodiment, the pump 554 and motor 556 are supplied as an assembly, such as model no. 50200 available from Thomas Pumps and Compressors of Shebogan, Wis.

The reservoir 558 in one embodiment is a four liter bottle, such as Nalgene® model no. 2125-4000 available from Nalge Nunc International of Rochester, N.Y. The reservoir size will be selected to suit a particular application and, as will be discussed in greater detail below, is typically substantially larger than the microfluidic volume to be pulled by the vacuum system 552. In addition, the reservoir material can be a metal, a polymer, glass, or combinations thereof. In particular, the reservoir material should be compatible with the sample. Also, the reservoir 558 should be capable of withstanding the pressures to which the reservoir 558 is exposed. For example, the reservoir 558 should be able hold a vacuum with minimal leakage and without collapsing.

The vacuum system 552 shown in FIG. 11E includes three valves, 560A, 560B, 560C (collectively 560), although it should be understood that more or fewer valves can be included to suit a particular application, for example, one valve could be used to control the entire vacuum system 552 or one valve per microchannel could be used to control the suction for each individual channel. The valves 560 shown are two position, three connection type solenoid valves, such as model no. LHDA1233115H available from the Lee Co. of Westbrook, Conn. The solenoids, which actuate the valves, are energized by 12 volt DC; however, other voltages can be used and the valves can be actuated hydraulically, pneumatically, or manually. Additionally, the valve type and configuration can be selected to suit a particular application. For example, the valves can be two position, two connection or two position, four connection.

FIG. 11F shows a flow cell 10 loaded into the sample loading apparatus 500 with the loading 48 and unloading block 60 in position and ready to be loaded with a sample or samples. As shown, the unloading block 60 has a plurality of apertures 66, each corresponding to one of the channels of the flow cell 10. The cover 512 includes a microfluidic handling system 518 fluidly coupled to the vacuum system 552. As best shown in FIG. 11G, the microfluidic handling system 518 includes a plurality of conduits 520. Each of the conduits 520 has an inlet 522 and is coupled to an isolation valve 560 as described above. After the flow cell 10 is properly positioned on the heater 515, the cover 512 is closed and a temperature sensor 519 contacts the flow cell 10 (FIGS. 11D and 11I). The temperature sensor 519 allows monitoring of the temperature of the flow cell 10 during heating and/or cooling cycles.

Provided the flow cell 10 has been properly positioned, each of the inlets 522 align with one of the apertures 66 of the unloading block 60. Once the cover 512 is closed, it can be secured in the closed position with a latch 526 as shown in FIGS. 11I and 11J to seal the loading 48 and unloading blocks 60 against the flow cell and the microfluidic handling system 518 against the unloading block 60.

FIG. 11H shows the sample loading apparatus 500 with the cover 512 in the closed position. The cover 512 includes a transparent window section 528 to allow visualization of the loading block 48 and the channels of the flow cell 10. As best shown in FIG. 11I, a portion of the window section 528 defines an opening 530 in the cover 512 through which the user can access the wells 54 after the cover 512 is closed. The window section 528 seals around the periphery of the loading block 48 while still allowing access to the wells 54 for sample loading. The cover 512 also includes a plurality of buttons 532a, 532b, 532c, etc. (collectively 532) corresponding to each of the channels in the flow cell 10. The buttons 532 are connected (either electrically or both other means) to the isolation valves 560. When depressed, the button 532 opens the valve to allow fluid communication between its corresponding microchannel and the vacuum system 552 such that a fluid (such as air or sample) can be pulled from the wells 54, into or through the microchannel and out of the flow cell 10 into the microfluidic handling system 518.

In operation, the user preconditions the flow cell 10 with a buffer to rehydrate the channels 12. This is accomplished by dispensing a microfluidic volume of buffer into the loading wells 54 either individually, in groups, or all of the wells 54 simultaneously. This may either be done robotically or manually using a single pipette or a multi-gang pipette. Performing such an operation robotically is described in Published U.S. Patent Application US 2007/0012113, filed Jul. 18, 2005 to Ulmer, which is incorporated herein by reference in its entirety. Once the buffer (or other liquid sample) is loaded into the wells 54, the user depresses one of more of the buttons 532 to activate the vacuum system which suctions the buffer through the conical loading wells 54, down through the inlet hole 14, and then into the channels 12. The user can monitor the loading process visually through the window section 528 and once the channels are filled, the user releases the button 532 to stop the flow of buffer into the channels 12. After waiting a predetermined amount of time, the user actuates the button(s) 532 again to evacuate the buffer from the flow cell 10.

Next, the user dispenses a microfluidic volume of a sample or samples into the loading wells 54 either individually or simultaneously. As described above, this may either be done either robotically, or manually using a single pipette or a multi-gang pipette. Once the sample is loaded into the wells 54 the user depresses one of more of the buttons 532 to activate the vacuum system 552 which pulls the sample through the conical loading wells 54, down through the inlet hole 14, and then into the channels 12. Once again the user can monitor the loading process visually through the window section 528 and release the button 532 to stop the flow of sample into the channels 12 once they are filled. After the channels are fully loaded, a quantity of unused sample may still remain in the loading wells 54. Although the volume of sample remaining in the loading wells 54 may be very small, in some applications, the user may want to retain as much sample as possible because of the inability to obtain more sample, or because of the cost of the sample. In such a situation, the user can aspirate the unused sample out of the loading wells 54 either manually or robotically with, for example, a pipette.

Before or after the sample is loaded, the user can program the apparatus 500 to a certain temperature, thus activating the heater 515 to warm the flow cell 10. The apparatus 500 includes a temperature control system, which includes the heater 515 and the temperature sensor 519 to control the temperature of the flow cell 10 to the temperature set by the user. The user waits the appropriate amount of time for the samples to hybridize, and then pumps out the sample from the flow cell 10 as described above. Each loading well 54 and corresponding channel 12 may be isolated from the adjacent loading wells and channels, so that multiple distinct samples can be loaded and analyzed simultaneously without cross-contamination. This process of loading and unloading additional buffer solutions or reagents can be repeated as necessary for the particular analysis being performed. Once the flow cell 10 has been evacuated for the final time, the user opens the cover 512 and removes the flow cell 10 which is now ready to be loaded into an apparatus for further analytical processes.

In various embodiments, the first substrate 18 or second substrate 30 can be treated to react with the microfluidic volumes being pulled through the flow cell 10. For example, a plurality of DNA strings can be adhered to surfaces of the channels 12 that are formed by the substrates 18, 30.

One application for a flow cell 10 as described herein includes performing single molecule sequencing. In this application, the flow cell 10 includes individual strands of DNA or RNA (the “template”) bound to channels 12 of the flow cell 10. The template can be bound to the channels 12 by any of a variety of means for binding DNA or RNA to a surface using, for example, biotin-avidin interactions or other suitable attachment chemistries. A primer is added that hybridizes to a portion of the DNA or RNA bound in the flow cell 10. Such an application is described in Published U.S. Patent Application US 2006/0012784, filed Nov. 16, 2004 to Ulmer, which is incorporated herein by reference.

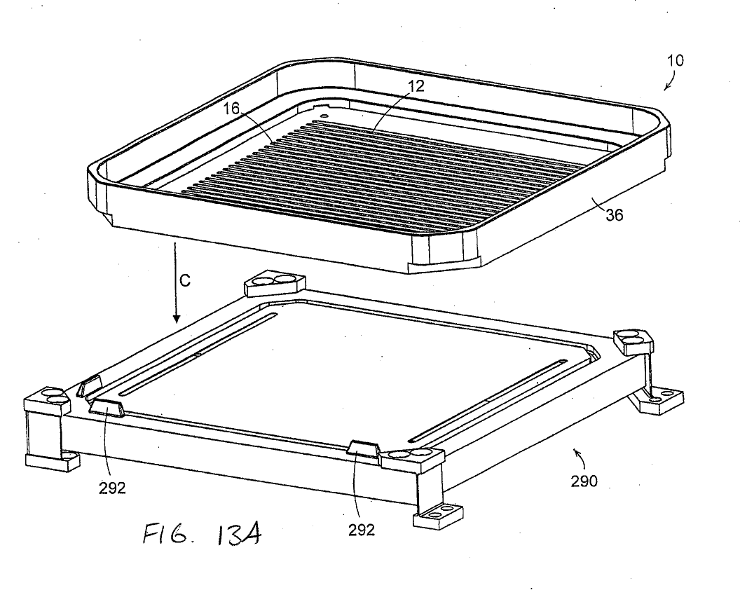

An example of an apparatus 200 that can be used to perform the processes described above is shown in FIG. 12. The apparatus 200 includes an optics section 210, a fluid handling section 220, a filter 230, a power supply 240, a laser control section 250, a bar code reader 260, a motor section 270, a central processing unit 280, and a flow chuck 290. After a flow cell, such as the flow cell 10, has been prepared for analysis, it may be loaded into the flow chuck 290 of the apparatus 200.

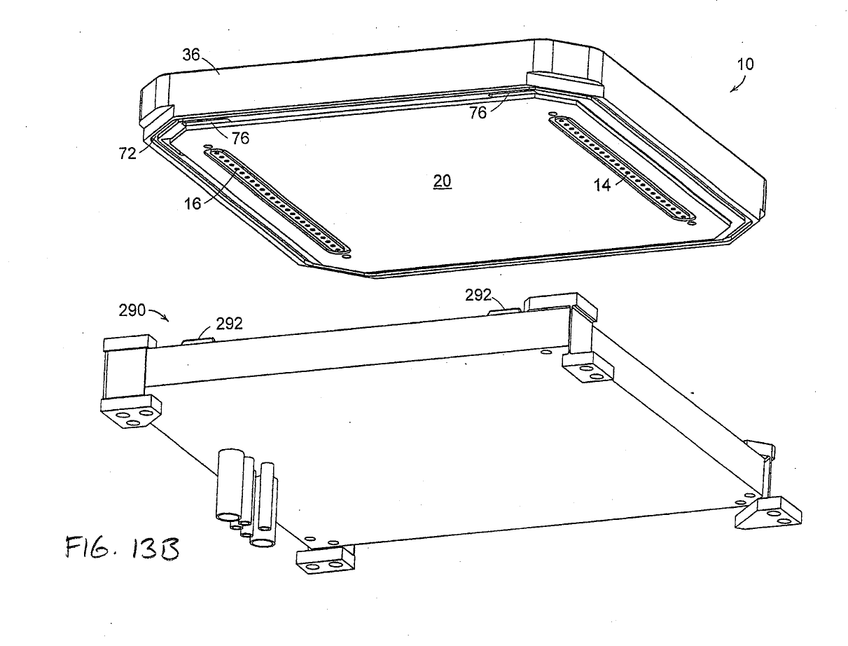

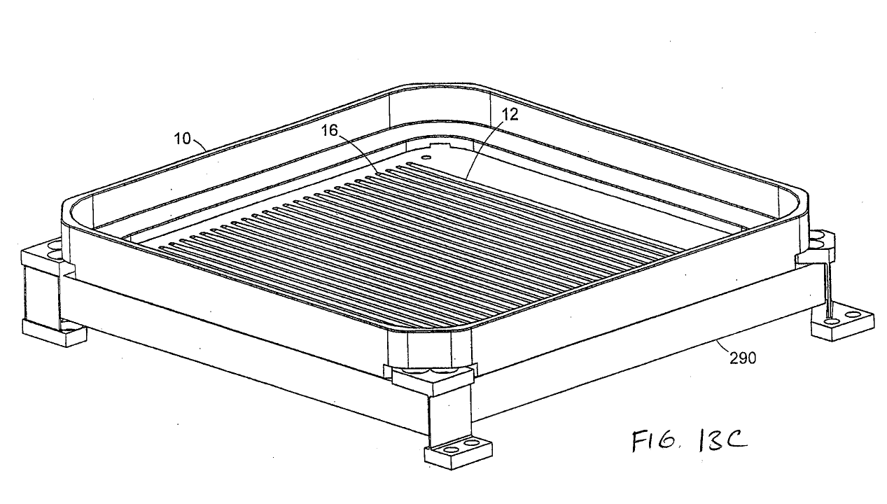

Referring now to FIGS. 13A-13C, the flow cell 10 is being loaded into the flow chuck 290. The flow cell 10 is inverted by the user such that the top surface 20 of the first substrate 18 is placed in contact with the flow chuck 290 in the direction indicated by line C in FIG. 13A. The flow cell 10 optionally includes a recess 72 formed near the periphery of the frame 36 (FIG. 13B) and a gasket or compressible tube (not shown) may be received in the recess 72 to create a tighter seal when the flow cell 10 is installed in the flow chuck 290. The flow chuck 290 optionally includes posts 292 that are received into slots 76 in the flow cell 10. The posts 292 are alignment features designed to ensure the flow cell 10 is mounted into the flow chuck 290 correctly. FIG. 13C shows the flow cell 10 mounted in the flow chuck 290 and ready for processing by the apparatus 200.

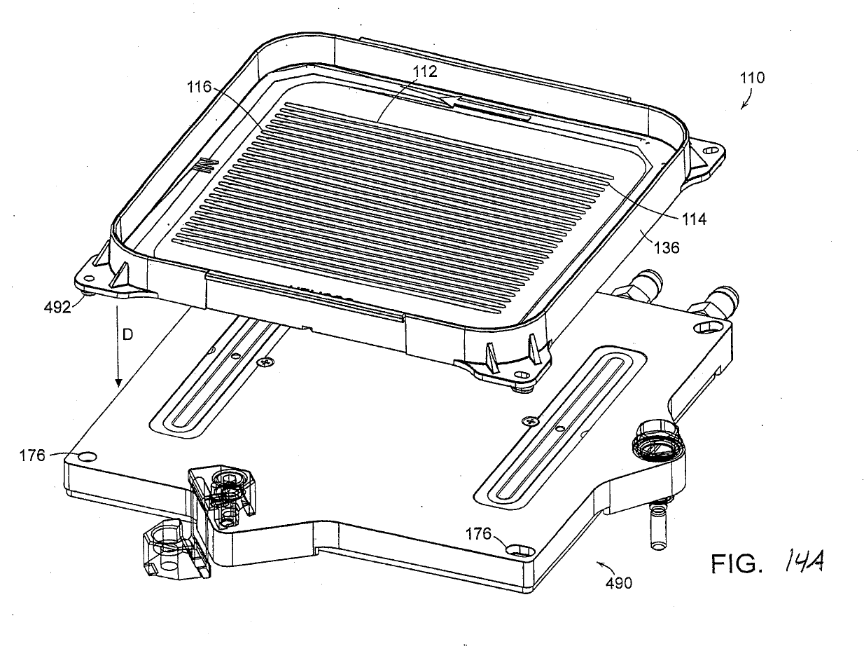

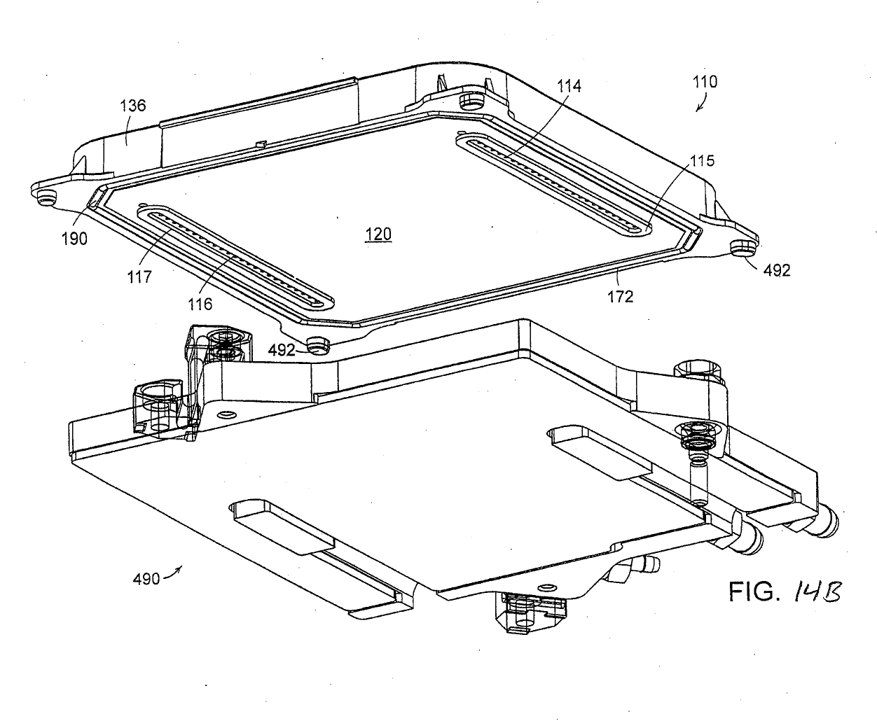

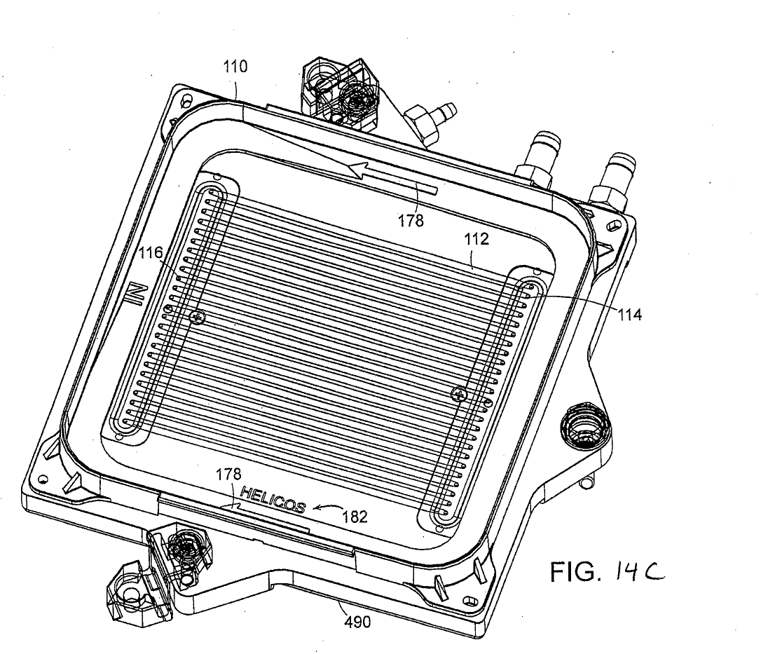

Referring now to FIGS. 14A-14C, the flow cell 110 is being loaded into an alternative embodiment of a flow chuck 490. The flow cell 110 is inverted by the user such that the top surface 120 of the first substrate 118 is placed in contact with the flow chuck 490 in the direction indicated by line D in FIG. 14A. As shown in FIG. 14B, the flow cell 110 has the compressible tube 190 disposed in the recess 172 to create a tighter seal when the flow cell 110 is installed in the flow chuck 490. In this embodiment, the flow cell 110 includes the posts 492 and the flow chuck 490 includes slots 176 to ensure proper positioning of the flow cell 110 in the flow chuck 490. The posts 492 also provide protection for the flow cell 110 so that the substrates 118, 130 doesn't break if accidentally dropped or put down improperly on the flow chuck 490. Additional alignment features of this embodiment of the flow cell 110 include arrows 178 and a logo 182. FIG. 14C shows the flow cell 110 mounted in the flow chuck 490 and ready for processing by the apparatus 200. Alternate embodiments of the flow cell may also include bar coding or other electromagnetic devices to ensure proper loading and to identify samples that are being analyzed.

Flow cells may be used individually, or optionally two or more flow cells may be combined together to analyze even more samples simultaneously. For example, FIG. 15 illustrates a dual flow cell 300, dual flow chuck 390 configuration. Although certain embodiments have been described, such description is for illustrative purposes only. Changes and variations may be made and are within the scope of this disclosure.

Microfluidic devices described above are useful for performing a variety of biochemical assays. In one embodiment, the biochemical assay comprises a sequencing-by-synthesis process. In one preferred embodiment, sequencing-by-synthesis is conducted on single, optically-isolated nucleic acid duplexes attached to a surface. Methods of the invention combine the reaction component of sequencing-by-synthesis in parallel with effective imaging in order to sequence target nucleic acids of interest with high efficiency and high accuracy.

In the embodiments described below, sequencing-by-synthesis is used as the exemplary biochemical assay. However, the flow cells of the present invention can be used for any biochemical assay that has a reaction component and a interrogation component, where the reaction and interrogation components are typically conducted in sequence in (or on) the same chamber.

Where the reaction time for the biochemical assay is about the same as the interrogation time, methods of the present invention comprises using a flow cell having a first and second area as described above. Where the biochemical assay is a sequencing-by-synthesis process, one or more nucleic acid duplexes comprising a template and a primer hybridized thereto are attached to a surface of a first imaging area of the flow cell. One or more nucleic acid duplexes comprising a template and a primer hybridized thereto are attached to a surface of a second imaging area of the flow cell. The duplexes comprise an optically-detectable label that is used to determine the position of individual duplexes on the surface. Once duplex positions are obtained, the reaction component (e.g., sequencing reaction) is performed on the first and second imaging areas of the flow cell. After completion of the sequencing reaction, the first imaging area is interrogated (e.g., imaged).

During this first round of the sequencing-by-synthesis process, the surfaces of both imaging areas are exposed to a labeled nucleotide triphosphate in the presence of a polymerase. Template strands that contain the complement of the labeled nucleotide immediately adjacent the 3′ terminus of the primer incorporate the added nucleotide. After a wash step to remove unincorporated nucleotide, the surface of the first imaging area is interrogated to determine which duplex positions have had a label added, those being the positions that have incorporated the added nucleotide, as described herein. While the first imaging area is being interrogated, the surface of the second imaging area can be stored in a suitable buffer to maintain the stability of the attached duplexes, for example in a neutral buffer such as a HEPES buffer.

After interrogation of the surface of the first imaging area is completed, the surface of the second imaging area is interrogated in a similar fashion. The surface of the second imaging area can be washed after storage and before interrogation. While the surface of the second imaging area is being interrogated, the sequencing reaction is performed on the surface of the first imaging area as described above. After interrogation, the added label can be removed. The surface of the first imaging area can be stored in a neutral buffer, as described above, until it is time to interrogate the surface of the first imaging area again.

After interrogation of the surface of the second imaging area is completed, the surface of the first imaging area is interrogated as described above. The surface of the first imaging area can be washed after storage and before interrogation. While the surface of the first imaging area is being interrogated, the sequencing reaction is performed on the surface of the second imaging area as described above. After interrogation, the added label can be removed. The surface of the second imaging area can be stored in a neutral buffer, as described above, until it is time to interrogate the surface of the second imaging area. In this manner, the reaction component and the interrogation component of the biochemical assay are performed in parallel using the same flow cell.

The cycle of performing sequencing-by-synthesis and interrogation can be repeated. After a sufficient number of reactions have been performed, the data set produced is a stack of image data for each imaging area that shows the linear results of the reaction component of the biochemical assay. For example, where the biochemical assay is a sequencing-by-synthesis process, after a sufficient number of nucleotides (determined by the desired read length as discussed below) have been exposed to the surface-bound templates of the first and second imaging areas, the data set produced is a stack of image data for each imaging area that shows the linear sequence of the individual duplex positions identified on the surface of that imaging area.

Where the reaction time required of the biochemical assay is greater than the interrogation time, the flow cell comprises at least two imaging areas, each having a surface, wherein biological molecules of interest are attached in multiple spots on each surface. For example, where the biochemical assay is a sequencing-by-synthesis process, as described above, duplexes are attached to the surfaces of each imaging area such that each surface has two or more spots where the duplexes are attached.

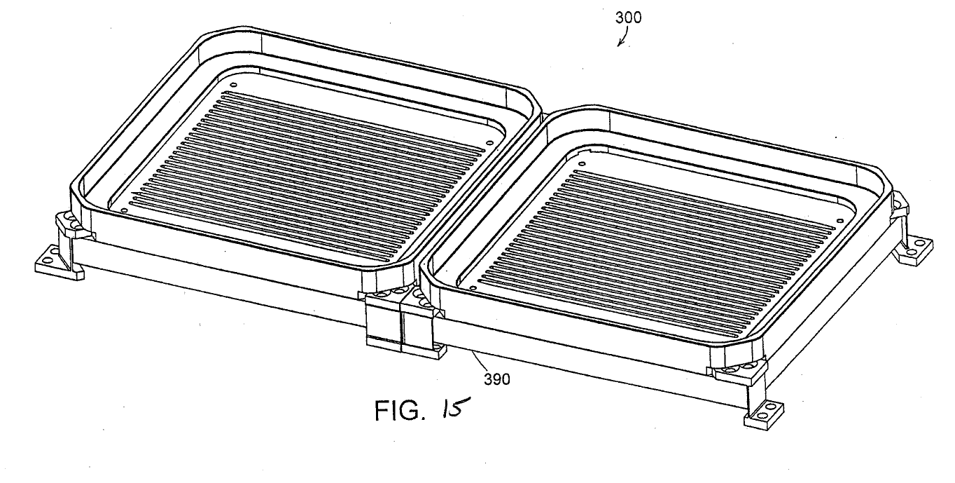

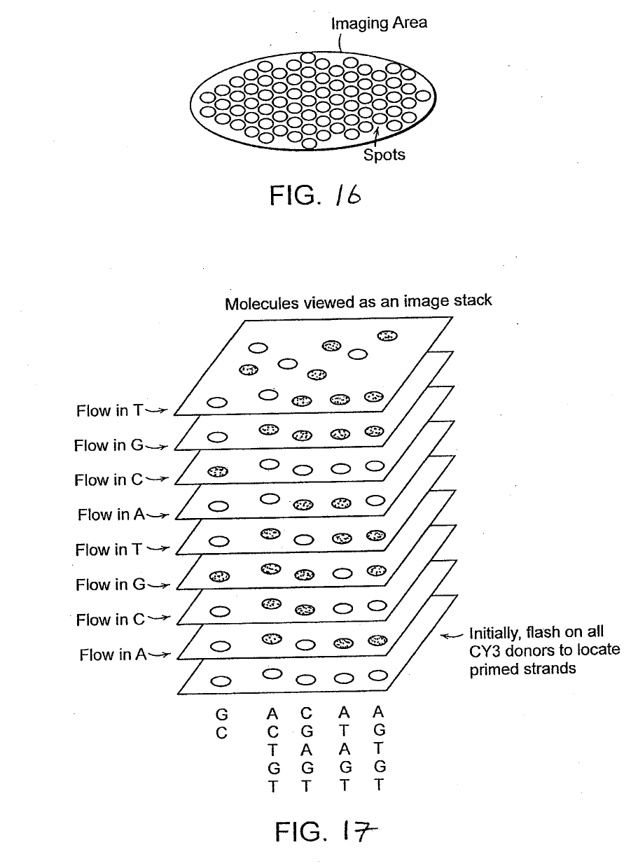

The number of spots per imaging area will depend upon the ratio of the reaction time to the interrogation time. For example, if the sequencing reaction takes three times as long as the interrogation, then the duplexes can be attached to each surface in three spots. Each spot is interrogated separately. Therefore, the total interrogation time per imaging area is the time it takes to interrogate each spot, multiplied by the number of spots per imaging area. The reaction time is the time it takes to perform the reaction component on one spot because they are processed simultaneously in the same imaging area. Therefore, the time it takes to interrogate all of the spots in one imaging area will approximate the amount of time it takes to complete the sequencing reaction for the other imaging area. FIG. 16 shows a schematic of multiple spots in an imaging area.

Where the reaction time of the biochemical assay is less than the interrogation time, then the flow cell comprises three or more imaging areas as described above. The method of using the flow cell comprising three or more imaging areas comprises attaching the biochemical molecules required for the particular biochemical assay to the surfaces of each of the imaging areas. For example, where the biochemical assay is a sequencing-by-synthesis process, duplexes as described above are attached to the surfaces of each of the imaging areas. Once duplex positions are obtained, the reaction component of the biochemical assay is performed simultaneously on each of the imaging areas of the flow cell.

In one embodiment, the surfaces of the imaging areas are exposed to a labeled nucleotide triphosphate in the presence of a polymerase. Template strands that contain the complement of the labeled nucleotide immediately adjacent the 3′ terminus of the primer incorporate the added nucleotide. After a wash step to remove unincorporated nucleotide, the surface of the first imaging area is interrogated in order to determine which duplex positions have a label added, those being the positions that have incorporated the added nucleotide. While the surface of the first imaging area is being interrogated, the surfaces of the other imaging areas can be maintained in a suitable buffer as described above. After interrogation of the surface of the first imaging area, the label can be removed.

Next, the surface of the second imaging area is interrogated and the reaction component of the biochemical assay (e.g., the sequencing reaction) is performed on the surface of the first imaging area as described above. After a wash step to remove unincorporated nucleotide, the surface of the first imaging area is stored, as described above, until it is time to interrogate the surface of the first imaging area. Thus, the interrogation (e.g., imaging) of the second imaging area is performed in parallel with the reaction component (e.g., sequencing) of the first imaging area. After interrogation of the surface of the second imaging area, the label can be removed.

Next, the surface of the third imaging area is interrogated and the reaction component of the biochemical assay (e.g., the sequencing reaction) is performed on the surface of the second imaging area as described above. After a wash step to remove unincorporated nucleotide, the surface of the second imaging area is stored, as described above, until it is time to interrogate the surface of the second imaging area. Thus, the interrogation (e.g., imaging) of the third imaging area is performed in parallel with the reaction component (e.g., sequencing) of the second imaging area. After interrogation of the surface of the third imaging area, the label can be removed.

The cycle of performing sequencing-by-synthesis and interrogation in parallel can be repeated. After a sufficient number of reactions have been performed the data set produced is a stack of image data for each imaging area that shows the linear results of the reaction component of the biochemical assay. For example, where the biochemical assay is a sequencing-by-synthesis process, after a sufficient number of nucleotides (determined by the desired read length as discussed below) have been exposed to the surface-bound templates of the imaging areas, the data set produced is a stack of image data for each imaging area that shows the linear sequence of nucleotides incorporated at each of the individual duplex positions identified on the surface of that imaging area.

The number of imaging areas can be increased, depending on the ratio of reaction time to interrogation time. Generally, the number of imaging areas can be the same as the fold difference between reaction time and interrogation time. Therefore, if the reaction takes twice as long as the interrogation, then the flow cell can comprise two imaging areas. If the reaction takes three times as long, then the flow cell can comprise three imaging areas; five imaging areas for a five fold difference, 10 imaging areas for a 10 fold difference, 20 imaging areas for a 20 fold difference, and so on.

Methods according to the invention provide de novo sequencing, re-sequencing, DNA fingerprinting, polymorphism identification, for example single nucleotide polymorphisms (SNP) detection, as well as applications for genetic cancer research. Applied to RNA sequences, methods according to the invention also are useful to identify alternate splice sites, enumerate copy number, measure gene expression, identify unknown RNA molecules present in cells at low copy number, annotate genomes by determining which sequences are actually transcribed, determine phylogenic relationships, elucidate differentiation of cells, and facilitate tissue engineering. Methods according to the invention are also useful to analyze activities of other biomacromolecules such as RNA translation and protein assembly.

Preferred methods for single molecule sequencing of nucleic acid templates comprise conducting a template-dependent sequencing reaction in which multiple labeled nucleotides are incorporated consecutively into a primer such that the accuracy of the resulting sequence is at least 70% with respect to a reference sequence. The primer is part of an optically-isolated substrate-bound duplex comprising a nucleic acid template having the primer hybridized thereto. The duplex is bound to the substrate such that the duplex is individually optically resolvable on the substrate.

As described herein, a plurality of labeled nucleotides are incorporated consecutively into one or more individual primer molecules. In some embodiments, at least three consecutive nucleotides, each comprising an optically-detectable label, are incorporated into an individual primer molecule. In other embodiments, at least 5, at least 10, at least 20, at least 30, at least 50, at least 100, at least 500, at least 1000 or at least 10000 consecutive nucleotides, each comprising an optically-detectable label, are incorporated into an individual primer molecule.

The accuracy of the resulting sequence is at least about 70% with respect to a reference sequence, between about 75% and about 90% with respect to a reference sequence, or between about 90% and about 99% with respect to a reference sequence. Preferably, the accuracy of the resulting sequence can be greater than about 99% with respect to a reference sequence. The reference sequence can be, for example, the sequence of the template nucleic acid molecule, if known, or the sequence of the template obtained by other sequencing methods, or the sequence of the a corresponding nucleic acid from a different source, for example from a different individual of the same species or the same gene from a different species.

Methods for single molecule nucleic acid sequencing also comprise incorporating at least three consecutive nucleotides, each comprising an optically-detectable label, into a primer. The primer is part of a template/primer duplex. The template, primer or both is/are attached to a solid substrate such that the duplex is individually optically resolvable.

In a particular embodiment of the invention, all four nucleotides are added during the biochemical component of each cycle, with each nucleotide containing a detectable label. In a highly-preferred embodiment of the invention, the label attached to added nucleotides is a fluorescent label. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′ 5″-dibromopyrogallol-sulfonaphthalein (Bromopyrogallol Red); 7-diethyl amino-3-(4′-isothiocyanatophenyl)-4-methylcoumarin; diethylenetriamine pentaacetate; 4,4′-diisothiocyanatodihydro-stilbene-2,2-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

A preferred surface for the imaging areas of the flow cells of the invention is an epoxide surface on a glass or fused silica slide or cover slip. However, any surface that has low native fluorescence is useful in the invention. Other surfaces include, but are not limited to, Teflon, polyelectrolyte multilayers, and others. The only requirement of a surface for use in the invention is that it has low native fluorescence and has the ability to bind nucleic acids, either directly or indirectly.

In a preferred embodiment, nucleic acid template molecules are attached to a substrate (also referred to herein as a surface) and subjected to analysis by single molecule sequencing as taught herein. Nucleic acid template molecules are attached to the surface such that the template/primer duplexes are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

In one embodiment, a substrate is coated to allow optimum optical processing and nucleic acid attachment. Substrates for use in the invention can also be treated to reduce background. Exemplary coatings include epoxides, and derivatized epoxides (e.g., with a binding molecule, such as streptavidin). The surface can also be treated to improve the positioning of attached nucleic acids (e.g., nucleic acid template molecules, primers, or template molecule/primer duplexes) for analysis. As such, a surface according to the invention can be treated with one or more charge layers (e.g., a negative charge) to repel a charged molecule (e.g., a negatively charged labeled nucleotide). For example, a substrate according to the invention can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Molecular Dynamics, Sunnyvale, Calif. In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase template molecule binding to the substrate or, at least, do not substantially impair template binding.

Various methods can be used to anchor or immobilize the nucleic acid template molecule to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11: 107-115, 1986. A preferred attachment is direct amine bonding of a terminal nucleotide of the template or the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

Any polymerizing enzyme may be used in the invention. A preferred polymerase is Klenow with reduced exonuclease activity. Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W.H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DPII/DP2 DNA polymerase II (Cann et al., 1998, Proc Natl Acad. Sci. USA 95:14250—>5).

Other DNA polymerases include, but are not limited to, ThermoSequenase®, 9°Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit. Rev Biochem. 3:289-347 (1975)).

In a preferred embodiment of the invention, direct amine attachment is used to attach primer, template, or both as duplex to an epoxide surface. The primer or the template comprises an optically-detectable label in order to determine the location of duplex on the surface. At least a portion of the duplex must be optically resolvable from other duplex on the surface. The surface is preferably passivated with a reagent that occupies portions of the surface that might, absent passivation, fluoresce. Optimal passivation reagents include amines, phosphate, water, sulfates, detergents, and other reagents that reduce native or accumulating surface fluorescence. Sequencing is then accomplished by presenting one or more labeled nucleotide in the presence of a polymerase under conditions that promote complementary base incorporation in the primer. In a preferred embodiment, one base at a time (per cycle) is added and all bases have the same label. There is a wash step after each incorporation cycle, and the label is either neutralized without removal or removed from incorporated nucleotides. After the completion of a predetermined number of cycles of base addition, the linear sequence data for each individual duplex is compiled. Numerous algorithms are available for sequence compilation and alignment as discussed below.

In resequencing, a preferred embodiment for sequence alignment compared sequences obtained to a database of reference sequences of the same length, or within 1 or 2 bases of the same length, from the target in a look-up table format. In a preferred embodiment, the look-up table contains exact matches with respect to the reference sequence and sequences of the prescribed length or lengths that have one or two errors (e.g., 9-mers with all possible 1-base or 2-base errors). The obtained sequences are then matched to the sequences on the look-up table and given a score that reflects the uniqueness of the match to sequence(s) in the table. The obtained sequences are then aligned to the reference sequence based upon the position at which the obtained sequence best matches a portion of the reference sequence. More detail on the alignment process is provided below in the Example.

In another embodiment of the invention, fluorescence resonance energy transfer (FRET) is used to generate signal from incorporated nucleotides in single molecule sequencing of the invention. FRET can be conducted as described in Braslaysky, et al., 100 PNAS: 3960-64 (2003), incorporated by reference herein. In one embodiment, a donor fluorophore is attached to the primer portion of the duplex and an acceptor fluorophore is attached to a nucleotide to be incorporated. In other embodiments, donors are attached to the template, the polymerase, or the substrate in proximity to a duplex. In any case, upon incorporation, excitation of the donor produces a detectable signal in the acceptor to indicate incorporation.

In another embodiment of the invention, nucleotides presented to the surface for incorporation into a surface-bound duplex comprise a reversible blocker. A preferred blocker is attached to the 3′ hydroxyl on the sugar moiety of the nucleotide. For example an ethyl cyanine (—OH—CH2CH2CN) blocker, which is removed by hydroxyl addition to the sample, is a useful removable blocker. Other useful blockers include fluorophores placed at the 3′ hydroxyl position, and chemically labile groups that are removable, leaving an intact hydroxyl for addition of the next nucleotide, but that inhibit further polymerization before removal.

In another embodiment, individually optically resolvable complexes comprising polymerase and a target nucleic acid are oriented with respect to each other for complementary base addition in a zero mode waveguide. In one embodiment, an array of zero-mode waveguides comprising subwavelength holes in a metal film is used to sequence DNA or RNA at the single molecule level. A zero-mode waveguide is one having a wavelength cut-off above which no propagating modes exist inside the waveguide. Illumination decays rapidly incident to the entrance to the waveguide, thus providing very small observation volumes. In one embodiment, the waveguide consists of small holes in a thin metal film on a microscope slide or cover slip. Polymerase is immobilized in an array of zero-mode waveguides. The waveguide is exposed to a template/primer duplex, which is captured by the enzyme active site. Then a solution containing a species of fluorescently-labeled nucleotide is presented to the waveguide, and incorporation is observed after a wash step as a burst of fluorescence.

Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-1 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

Nucleic acid template molecules include deoxyribonucleic acid (DNA) and/or ribonucleic acid (RNA). Nucleic acid template molecules can be isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. Biological samples of the present invention include viral particles or preparations. Nucleic acid template molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid template molecules may also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, or genomic DNA.

Nucleic acid obtained from biological samples typically is fragmented to produce suitable fragments for analysis. In one embodiment, nucleic acid from a biological sample is fragmented by sonication. Nucleic acid template molecules can be obtained as described in U.S. Patent Application 2002/0190663 A1, published Oct. 9, 2003, the teachings of which are incorporated herein in their entirety. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, individual nucleic acid template molecules can be from about 5 bases to about 20 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulfonate. It is contemplated also that urea may be added with or without another detergent or surfactant.

Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

Other aspects and advantages of the invention are apparent to the skilled artisan upon consideration of the following drawings, detailed description of the invention and example.

The invention is described in the context of a template-dependent sequencing-by-synthesis reaction. Generally, the reaction comprises attaching template/primer duplex to an epoxide surface of two or more imaging areas as described above. Parallel sequencing-by-synthesis reactions are conducted on the surface of one imaging area using optical detection of incorporated nucleotides of a second imaging area followed by sequence compilation of both imaging areas. Either de novo sequencing or resequencing of a reference sequence is possible using methods of the invention. Partial sequencing can also be conducted using methods of the invention as will be apparent to those of ordinary skill in the art upon consideration of the disclosure herein. In a preferred embodiment, single duplex molecules are sequenced in parallel by placing them on the epoxide surface and confirming their locations. In that embodiment, only duplex that is optically-isolated from other duplex is used for sequencing. Single duplex sequencing avoids the requirement for amplification of template nucleic acids. Amplified, bulk sequencing can also be used in methods of the invention.

In general, epoxide-coated glass surfaces are used for direct amine attachment of templates, primers, or both. Amine attachment to the termini of template and primer molecules is accomplished using terminal transferase as described below. Primer molecules can be custom-synthesized to hybridize to templates for duplex formation. In a preferred embodiment, as described below, template fragments are polyadenylated and a complementary poly(dT) oligo is used as the primer. In this way, surfaces having previously-bound universal primers were prepared for sequencing heterogeneous fragments obtained from genomic DNA or RNA.

Sequencing according to the invention combines sample preparation, surface preparation and oligo attachment, interrogation, and analysis in order to achieve high-throughput sequence information. In one embodiment, optically-detectable labels were attached to templates that were attached directly to an epoxide surface. Individual template molecules were imaged in order to establish their positions on the surface. Then, primer was added to form duplex on the surfaces, and individual nucleotides containing an optical label were added in the presence of polymerase for incorporation into the 3′ end of the primer at a location in which the added nucleotide is complementary to the next-available nucleotide on the template immediately 5′ (on the template) of the 3′ terminus of the primer. Unbound nucleotide is washed out, scavenger is added, and the surface is imaged. Optical signal at a position previously noted to contain a single duplex (or primer) is counted as an incorporation event. Label is removed and the remaining linker is capped and the system is again washed. The cycle is repeated with the remaining nucleotides. A full-cycle is conducted as many times as necessary to complete sequencing of a desired length of template. Once the desired number of cycles is complete, the result is a stack of images as shown in FIG. 17 represented in a computer database. For each spot on the surface that contained an initial individual duplex, there will be a series of light and dark image coordinates, corresponding to whether a base was incorporated in any given cycle. For example, if the template sequence was TACGTACG and nucleotides were presented in the order CAGU(T), then the duplex would be “dark” (i.e., no detectable signal) for the first cycle (presentation of C), but would show signal in the second cycle (presentation of A, which is complementary to the first T in the template sequence). The same duplex would produce signal upon presentation of the G, as that nucleotide is complementary to the next available base in the template, C. Upon the next cycle (presentation of U), the duplex would be dark, as the next base in the template is G. Upon presentation of numerous cycles, the sequence of the template would be built up through the image stack. The sequencing data are then fed into an aligner as described below for resequencing, or are compiled for de novo sequencing as the linear order of nucleotides incorporated into the primer.

There are numerous alternatives to practice of the invention. For example, the inventors have attached primer via a direct amine attachment to an epoxide surface, but have also attached template first and have attached duplex (i.e., duplex was formed first and then attached to the surface). The inventors have also functionalized an epoxide surface with one member of a binding pair, the other member of the binding pair being attached to the template, primer, or both for attachment to the surface. For example, the surface was functionalized with stretptavidin, and biotin was attached to the termini of either the template, the primer, or both.

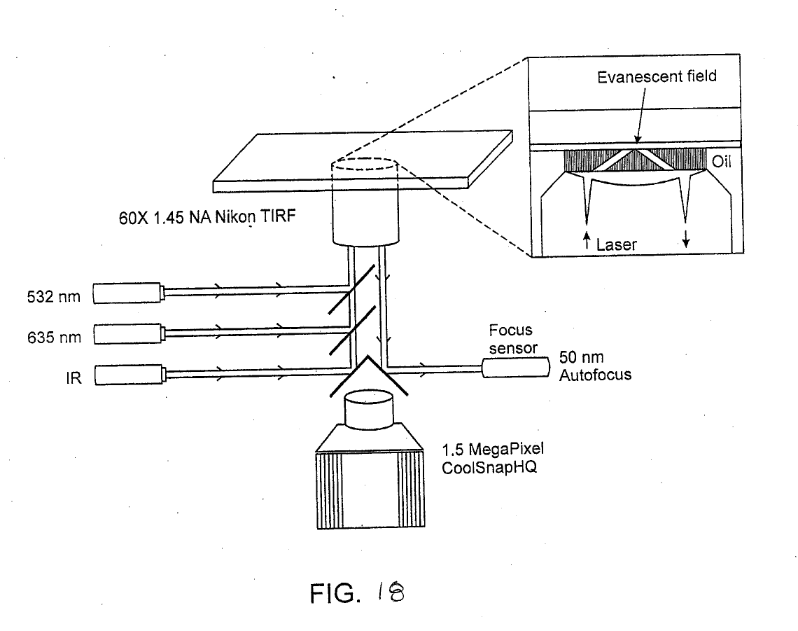

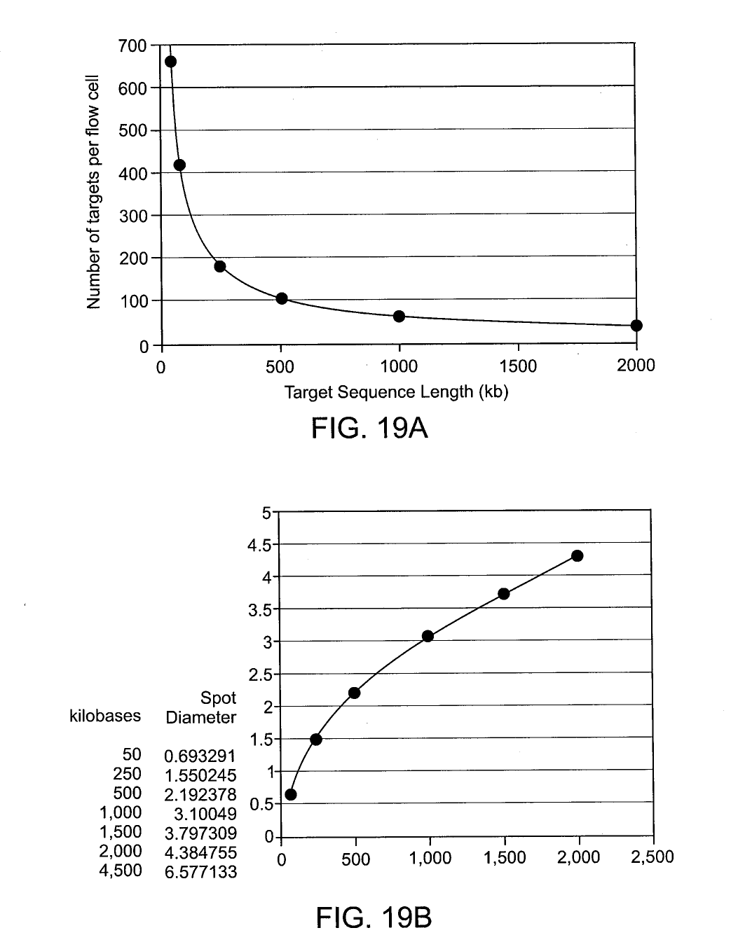

The imaging system to be used in the invention can be any system that provides sufficient illumination of the sequencing surface at a magnification such that single fluorescent molecules can be resolved. The imaging system used in the example described below is shown in FIG. 18. In general, the system comprised three lasers, one that produces “green” light, one that produces “red” light, and in infrared laser that aids in focusing. The beams are transmitted through a series of objectives and mirrors, and focused on the image as shown in FIG. 18. Imaging is accomplished with an inverted Nikon TE-2000 microscope equipped with a total internal reflection objective (Nikon).

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

The disclosed embodiments are exemplary. The invention is not limited by or only to the disclosed exemplary embodiments. Also, various changes to and combinations of the disclosed exemplary embodiments are possible and within this disclosure, Those skilled in the art will recognize, or be able to ascertain using no more than routine experimentation, many equivalents of the specific embodiments of the invention described herein. Such equivalents are intended to be encompassed by the following claims.

What is claimed is:

1. A system for loading a sample into a flow cell comprising:
a housing adapted to receive a flow cell, the flow cell defining a plurality of individually isolated channels defining, an inlet port and an outlet port associated with each of the channels;
a cover adapted to secure said flow cell in said housing; and
a vacuum source for pulling a fluid volume through said flow cell.

2. The system of claim 1 further comprising a loading block defining a plurality of wells and being removably attachable to the housing and in fluid communication with the flow cell, such that each of the inlet ports corresponds to one of the wells.

3. The system of claim 1 further comprising an outlet block being removably attachable to the housing and flow cell in registration with the outlet ports.

4. The system of claim 1 wherein the outlet ports of the flow cell are fluidly coupled to the vacuum source.

5. The system of claim 1 wherein the chamber further comprises heater.

6. The system of claim 1 wherein the chamber further comprises springs to allow compression when the cover is in a closed position.

7. The system of claim 1 wherein the chamber further comprises a compressible material to allow compression when the cover is in a closed position.

8. The system of claim 1 wherein the cover includes a window section for a user to visualize the flow cell when the cover is in a closed position.

9. The system of claim 1 wherein the cover further comprises a microfluidic handling system fluidly coupled to the vacuum source.

10. The system of claim 1 wherein the cover further comprises a temperature sensor that contacts the flow cell when the cover is in a closed position.

11. The system of claim 1 further comprising a latch to secure the cover in a closed position.

12. A method of loading a sample into a flow cell comprising:
providing a flow cell defining a plurality of individually isolated channels through which fluid can flow, the flow cell defining an inlet port and an outlet port for each of the channels;
placing a loading block defining a plurality of wells on the flow cell such that each of the wells correspond to one of the inlet ports;
placing an unloading block on the flow cell in registration with the outlet ports;
inserting the flow cell into a sample loading apparatus;
placing a sample in at least one of the wells of the loading block;
activating a vacuum source fluidly coupled to the outlet ports to pull the sample into the channel.

13. The method of claim 12, further comprising monitoring the sample flowing into the channel.

14. The method of claim 13, further comprising deactivating the vacuum source after the channel is filled with the sample.

15. The method of claim 14, further comprising aspirating the remaining sample from the well.

16. The method of claim 12, further comprising deactivating the vacuum source after the channel is filled with the sample.

17. The method of claim 16, further comprising waiting a predetermined period of time for the samples to hybridize.

18. The method of claim 17, further comprising reactivating the vacuum source to evacuate the sample from the channel.

19. The method of claim 16, further comprising heating the flow cell to a predetermined temperature.

20. The method of claim 19, further comprising waiting a predetermined period of time for the samples to hybridize.

21. The method of claim 20, further comprising reactivating the vacuum source to evacuate the sample from the channel.

22. The method of claim 12, further comprising aspirating the remaining sample from the well.
